SEQUENCE LISTING (19 sequences)

SEQ ID NO: 1 - protein, 136 aa, Homo sapiens [sequence omitted]

SEQ ID NO: 2 - protein, 19 aa, synthetic construct
IKEFLQRFIHIVQSIINTS

SEQ ID NO: 3 - protein, 21 aa, synthetic construct
PKEFLERFVHLVQMFIHQSLS

SEQ ID NO: 4 - protein, 10 aa, synthetic construct
KVTAMKCFLL

SEQ ID NO: 5 - protein, 122 aa, synthetic construct
EVRLMASGGGLVQPGGSLRLSCAASEFTFSNYAMSWVRQAPGKGLEWVATISRGGDYTYYPDSVKGRFTISRDNSKNTLYLQMNSLRAEDTAVYYCARRVSMITGGWAMDYWGQGTLVTVSS

SEQ ID NO: 6 - protein, 122 aa, synthetic construct
EVQLVESGGGLVQPGGSLRLSCAASEFTFSNYAMSWVRQAPGKGLEWVATISRGGDYTYYPDSVKGRFTISRDNSKNTLYLQMNSLRAEDTAVYYCARRVSMITGGWAMDYWGQGTLVTVSS

SEQ ID NO: 7 - protein, 122 aa, synthetic construct
EVRLMASGGGLVQPGGSLRLSCAASEFTFSNYAMSWVRQAPGKGLEWVATISRGGDYTYYPDTVKGRFTISRDNSKNTLYLQMNSLRAEDTAVYYCARRVSMITGGYAMDYWGQGTLVTVSS

SEQ ID NO: 8 - protein, 122 aa, synthetic construct
EVQLVESGGGLVQPGGSLRLSCAASEFTFSNYAMSWVRQAPGKGLEWVATISRGGDYTYYPDSVKGRFTISRDNSKNTLYLQMNSLRAEDTAVYYCARRVSMITGGYAMDYWGQGTLVTVSS

SEQ ID NO: 9 - protein, 112 aa, synthetic construct
DVVMTQSPLSLPVTLGQPASISCRSSQSIVDITGNTYLEWYQQRPGQSPRLLIYKVFNRFSGVPDRFSGSGSGTDFTLKISRVEAEDVGVYYCFQDSFVPYTFGQGTKLEIK

SEQ ID NO: 10 - protein, 467 aa, synthetic construct
MGWTLVFLFLLSVTAGVHSEVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 234 aa, synthetic construct
MVSSAQFLGLLLLCFQGTRCEIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 119 aa, synthetic construct
QVQLQESGPGLVKPSGTLSLTCAVSGGSISSSNWWSWVRQPPGKGLEWIGEIYHYGYTNYNPSLKSRVTISVDKSKNQFSLKLSSVTAADTAVYYCAREGIGWPSFDYWGQGTLVTVSS

SEQ ID NO: 13 - protein, 108 aa, synthetic construct
SSELTQDPAASVALGQTVRITCQGDTLRSYYASWYQQKPGQAPILVIYGKNNRPSGIPDRFSGSSSGNTASLTITGAQAEDEADYYCNSRDLSGKNLVFGGGTKLTVL

SEQ ID NO: 14 - protein, 141 aa, synthetic construct
MKLWLNWVFLLTLLHGIQCEVQLVESGGGLVQPGGSLRLSCAASGFTFSDFYMEWVRQAPGKGLEWIAAASRNKANDYTTEYSASVKGRFIVSRDDSKNSLYLQMNSLKTEDTAVYYCARSYYRYDGMDYWGQGTTVTVSS

SEQ ID NO: 15 - protein, 128 aa, synthetic construct
MDFQVQIFSFLLISASVIVSRGEIVLTQSPATLSLSPGERATLSCSAISSVSYMYWYQQKPGQAPRLLIYDTSNLVSGVPARFSGSGSGTDYTLTISSLEPEDFAYYYCQQWNTYPYTFGGGTKVEIK

SEQ ID NO: 16 - protein, 452 aa, synthetic construct
EVQLVESGGGLVQPGGSLRLSCAASEFTFSNYAMSWVRQAPGKGLEWVATISRGGDYTYYPDSVKGRFTISRDNSKNTLYLQMNSLRAEDTAVYYCARRVSMITGGWAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 219 aa, synthetic construct
DVVMTQSPLSLPVTLGQPASISCRSSQSIVDITGNTYLEWYQQRPGQSPRLLIYKVFNRFSGVPDRFSGSGSGTDFTLKISRVEAEDVGVYYCFQDSFVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 452 aa, synthetic construct
EVRLMASGGGLVQPGGSLRLSCAASEFTFSNYAMSWVRQAPGKGLEWVATISRGGDYTYYPDTVKGRFTISRDNSKNTLYLQMNSLRAEDTAVYYCARRVSMITGGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 452 aa, synthetic construct
EVQLVESGGGLVQPGGSLRLSCAASEFTFSNYAMSWVRQAPGKGLEWVATISRGGDYTYYPDSVKGRFTISRDNSKNTLYLQMNSLRAEDTAVYYCARRVSMITGGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2026/0297174 A1
(43) Pub. Date: Oct. 1, 2026

(54) IL-15 INHIBITORS USEFUL FOR THE TREATMENT OF ATOPIC DERMATITIS

(71) Applicant: NOVARTIS PHARMA AG, Basel (CH)

(72) Inventors: Alain VICARI, Annecy (FR); Yolande CHVATCHKO MISSOTTEN, Confignon (CH)

(21) Appl. No.: 19/100,893

(22) PCT Filed: Aug. 3, 2023
(86) PCT No.: PCT/EP2023/071573

§ 371 Date: Feb. 3, 2025

(30) Foreign Application Priority Data
Aug. 4, 2022 (EP) 22188873.8

(51) Int. Cl. C07K 16/24; A61K 31/4985; A61K 31/502; A61K 31/519; A61K 31/573; A61K 39/00; A61K 45/06; A61P 17/00; A61P 29/00; C07K 16/28

(52) U.S. Cl. CPC C07K 16/244; A61K 31/4985; A61K 31/502; A61K 31/519; A61K 31/573; A61K 45/06; A61P 17/00; A61P 29/00; C07K 16/2866; A61K 2039/507; C07K 2317/24; C07K 2317/51; C07K 2317/515; C07K 2317/56; C07K 2317/76

(57) ABSTRACT

The present invention relates to the use of agents able to neutralize the activity of interleukin-15 in the treatment of atopic dermatitis. In particular, the invention relates to IL-15 inhibitors for use in the prevention and/or treatment of atopic dermatitis.

### FIELD OF THE INVENTION

The present invention relates to the use of agents able to neutralize the activity of interleukin-15 in the treatment of atopic dermatitis.

### BACKGROUND OF THE INVENTION

Atopic dermatitis (AD), also known as eczema, is a chronic major inflammatory skin disease. It is characterized by dry skin, recurrent flares, and pruritus (itch).

The prevalence of AD varies widely across the globe due to regional, country-specific, age group, and data-capturing methodological differences, affecting 0.2% to 36% of the paediatric population (ages <18 years (Bylund et al., 2002, Acta Derm Venereol., 100(12):adv00160. https://doi.org/10.2340/00015555-3510; Eichenfleld et al., 2022, Pediatr Drugs 24, 293-305, https://doi.org/10.1007/s40272-022-00499-x). A recent international, Web-based survey found the prevalence of previously diagnosed and active AD ranged from 2.1% to 4.9% and that the prevalence of AD in adults is similar to that in adolescents and is stable throughout adulthood (Silvenberg et al., 2020, Med. Clin. N. Am., 104,157-176, https://doi.org/10.1016/j.mcna.2019.08.009). Globally, AD prevalence is often highest in high-income countries. It has also been widely observed that AD shows a female preponderance, particularly in adolescence and adulthood (Silvenberg et al., 2020, supra).

The condition usually starts in childhood and may continue through adulthood with varying severity over the years. AD is a complex, multifactorial diseases that involves:
i) a strong genetic component: AD can be considered as a familial disease and several susceptibility genes have been identified.
ii) Environmental exposure including irritants and pruritogens, pathogens, climate factors, ultraviolet radiation, outdoor and indoor air pollutants, tobacco smoke exposure, water hardness, urban vs. rural living, diet, breastfeeding, probiotics and prebiotics. Some of these factors may explain the rise of atopic dermatitis in the last decades
iii) Altered permeability of the skin. About 30% of the people with atopic dermatitis have mutations in the gene for the production of filaggrin (FLG), which increase the risk for early onset of atopic dermatitis and developing asthma. Filaggrin plays an important role in keeping the skin surface slightly acidic, hence giving it anti-microbial effects.

These different components associate and synergize to drive a complex immune pathology involving multiple pathways, cytokines and cell types, ultimately responsible for the signs and symptoms of atopic dermatitis. Evidence suggests that several cytokines, such as the T helper 2 cytokines interleukin 4 (IL-4) and closely related IL-13, are important components of the pathogenesis of AD. Indeed, an antibody (dupilumab) targeting the IL-4 receptor alpha subunit (IL-4Rα) that is shared between IL-4 and IL-13 was the first biologic approved for the treatment of Atopic Dermatitis in Europe and the USA alone or in combination with corticosteroids (Strowd et al., 2017, The Lancet 389, 2265-2266. https://doi.org/10.1016/S0140-6736(17)31192-3) and more recently an antibody targeting IL-13 (tralokinumab) has also been approved in Europe and the USA alone or in combination with corticosteroids (Wollenberg et al., 2021, Br J Dermatol., 184(3): 437-449, doi: 10.1111/bjd.19574; Silverberg et al., 2021, Br J Dermatol, 184(3):450-463, doi: 10.1111/bjd.19573). Nevertheless, many patients do not respond or inadequately, leaving a large remaining large unmet medical need in atopic dermatitis, for efficient and safe treatments, and there are several products in development targeting other pathways than IL-4/IL-13.

Interleukin-15 (IL-15), also known as MGC9721, is a cytokine that is well known as a regulator of natural killer (NK) and T cell activation, survival and proliferation (Waldmann et al., 2020, J. Exp. Med., 217, e20191062, https://doi.org/10.1084/jem.20191062). But it is also a proven modulator of other cells such as B cells, monocytes and eosinophils (Gill et al., 2009, Cell. Immunol., 258, 59-64, https://doi.org/10.1016/j.cellimm.2009.03.010; Mohamadzadeh et al., 2001, Journal of Experimental Medicine, 194, 1013-1020, https://doi.org/10.1084/jem.194.7.1013; Hoontrakoon et al., 2002, Am. J. Respir. Cell Mol. Biol., 26, 404-412, https://doi.org/10.1165/ajrcmb.26.4.4517). This cytokine and interleukin 2 (IL-2) share many biological activities, consistent with their shared receptor signalling components (IL-2/15Rβ and IL-2/15Rγc). However, specificity for IL-15 versus IL-2 is provided by unique private α-chain receptor that completes the IL-15Rαβγ hetero-trimeric high-affinity receptor complex and thereby allows differential responsiveness depending on the ligand and high-affinity receptor expressed (Fehniger and Caligiuri, 2001, Blood 97, 14-32

A deleterious role for a dysregulation of IL-15 expression has been demonstrated in autoimmune diseases such as rheumatoid arthritis, psoriasis, celiac disease, eosinophilic esophagitis, alopecia areata and vitiligo. This is based on the observation that compounds that neutralize IL-15 signaling could reduce clinical and diseases features in animal models of those diseases (Villadsen et al., 2003, J. Clin. Invest. 112, 1571-1580, https://doi.org/10.1172/JCI18986; Sestak et al., 2018, Front. Immunol. 9, 1603, https://doi.org/10.33891/fimmu.2018.01603; Lähdeaho et al., 2019, Lancet Gastroenterol Hepatol 4, 948-959; Vicari et al., 2017, MAbs 9, 927-944; Xing et al., 2014, Nature Medicine 20, 1043-1049, https://doi.org/10.1038/nm.3645; Richmond et al., 2018, Sci Transl Med 10, eaam7710, https://doi.org/10.1126/scitranslmed.aam7710) and in certain cases in clinical trials (Baslund et al., 2005, Arthritis Rheum. 52, 2686-2692, https://doi.org/10.1002/art.21249; Lähdeaho et al., 2019, supra). On the other hand, increased IL-15 expression might not be deleterious. For example, in viral diseases, increased IL-15 expression has been linked to improved viral clearance (Verbist et al., 2011, J. Immunol. 186, 174-182. https://doi.org/10.4049/jimmunol.1002613).

There have been several reports describing increased expression of IL-15, or its receptor, in the skin and/or blood of atopic dermatitis human patients (Orteu et al., 2000, Clin Exp Immunol., 122, 150-156; Karlen and Simon, 2020, Int. Arch. Allergy Immunol., 181, 417-421) or dogs (Mazrier et al., 2022, Vet. Dermatol., 33, 131-e38. https://doi.org/10.1111/vde.13044).

On the contrary, it has been also suggested that reduced IL-15 expression in humans could contribute to the pathogenesis of atopic dermatitis progression (Ong et al., 2002, J. Immunol., 168, 505-510). Further, a deficiency in blood Natural Killer (NK) cells was reported in atopic dermatitis patients, while skin from atopic dermatitis patients is enriched in activated NK cells (Mack et al., 2020, Sci. Transl. Med., 12, eaay1005. https://doi.org/10.1126/scitranslmed.aay1005). Given that IL-15 is an important factor for NK cell survival and activation, the authors suggested that providing IL-15 agonist molecules would have a beneficial effect in atopic dermatitis. To further support this hypothesis, they treated mice in the MC903 atopic dermatitis model (induced by topical application of a vitamin D3 analog) with an IL-15 agonist molecule and saw an improvement of clinical scores and other atopic dermatitis features, that were dependent upon NK cells (Mack et al., 2020, supra).

In conclusion, it is unclear from the existing art to determine whether IL-15 over-expression in atopic dermatitis is detrimental or beneficial.

Atopic dermatitis is the most common chronic inflammatory skin disease worldwide, with still limited therapeutic options. Therefore, given the substantial burden that AD places on affected subjects, there is a need for better understanding the underlying causes of AD and finding new agents having substantial beneficial effect in the management and treatment of AD.

### SUMMARY OF THE INVENTION

The present invention is mainly directed towards the use of agents that are able to inhibit interleukin-15 for the treatment of atopic dermatitis, in undefined global patient population, as well as in atopic dermatitis patients not responding to current treatments such as anti-IL-4R, anti-IL-4 or anti-IL-13 antibodies or to corticoid treatments.

According to a first embodiment, is provided an interleukin-15 inhibitor for use in the treatment of atopic dermatitis.

According to another embodiment, is provided the use of an interleukin-15 inhibitor for the preparation of a pharmaceutical composition for the treatment of atopic dermatitis.

According to another aspect of the invention is provided a method of preventing and/or treating atopic dermatitis in a subject, said method comprising administering in a subject in need thereof a therapeutically effective amount of an interleukin-15 inhibitor or pharmaceutical composition thereof.

According to another aspect of the invention is provided a pharmaceutical composition comprising an interleukin-15 inhibitor and an agent useful in the treatment of atopic dermatitis and a pharmaceutically acceptable carrier, diluent or excipient thereof.

Other features and advantages of the invention will be apparent from the following detailed description.

### DESCRIPTION OF THE FIGURES

FIG. 1: Increase of IL-15 (A) and IL-15Rα (B) gene expression in ex vivo human atopic dermatitis model as analyzed by RNAseq (relative units) in human skin explants 48 hours in culture with control or Th2-polarizing medium as described in Example 1. Symbols represent individual explants and groups mean are represented as bar plots +/− Standard Deviation.

FIG. 2: Induced IL-15 (A) and IL-15Rα (B) gene expression in ex vivo human atopic dermatitis model as analyzed by RNAseq (relative units) in human skin explants 48 hours in culture with control or Th2-polarizing medium or Th2-polarizing medium alone or with anti-IL-4R antibody or the corticoid betamethasone as described in Example 1. Symbols represent individual explants and groups mean are represented as bar plots +/− Standard Deviation.

FIG. 3: Increased skin IL-15 and IL-15Ra expression in humanized atopic dermatitis model as analyzed in human skin xenotransplants two weeks after injection of Th2-polarized PBMC (Th2) or LPS-activated Th2-polarized PBMC (Activated Th2) as described in Example 2. In the epidermis, IL-15 (A) and IL-15Rα (C) expression was quantified as mean intensity signal, while in the dermis IL-15 expression (B) was quantified as number of IL-15 positive cells per 100 dermal area units. Symbols represent individual xenografted mouse (grafts/PBMC from 2 separate human donors) and groups are represented as violin plots showing group median and quartiles.

FIG. 4: Effects of an anti-IL-15 antibody on clinical score measured and was analyzed in human skin xenotransplants as described in Example 2 at the time of injection of LPS-activated Th2-polarized PBMC (pre-treatment) and then after 2 weeks and 4 weeks treatment with various antibodies. As indicated in the x axis, the IgG1 group was injected during the 4 weeks with control isotype antibody, the anti-IL-15 group with anti-IL-15 antibody, and the IgG1+anti-IL-15 group with control isotype antibody for 2 weeks then anti-IL-15 antibody for 2 weeks. Symbols represent individual xenografted mouse and groups are represented as violin plots showing group median and quartiles.

FIG. 5: Anti-IL-15 antibody effect on the epidermis properties in humanized atopic dermatitis model as described in Example 2. A: Epidermal thickness of in human skin xenotransplants analyzed 4 weeks after injection of LPS-activated Th2-polarized PBMC in various antibody treatment groups. As indicated in the x axis, the IgG1 group was injected during the 4 weeks with control isotype antibody, the anti-IL-15 group with anti-IL-15 antibody, and the IgG1+anti-IL-15 group with control isotype antibody for 2 weeks then anti-IL-15 antibody for 2 weeks. Symbols represent individual xenografted mouse and groups are represented as violin plots showing group median and quartiles. B: Epidermal proliferation of in human skin xenotransplants was analyzed 4 weeks after injection of LPS-activated Th2-polarized PBMC in various antibody treatment groups by measuring the expression of Ki67 in the basal layer of human epidermis. As indicated in the x axis, the IgG group was injected during the 4 weeks with control isotype antibodies (due to inter-animal variability and smaller n per group, IgG1 and IgG4 groups were pooled), the anti-IL-15 group with anti-IL-15 antibody, and the IgG+anti-IL-15 group with control isotype antibody for 2 weeks then anti-IL-15 antibody for 2 weeks. Symbols represent individual xenografted mouse and groups are represented as violin plots showing group median and quartiles; C: Epidermal Filaggrin (FLG) in human skin xenotransplants analyzed 4 weeks after injection of LPS-activated Th2-polarized PBMC by immunofluorescence. As indicated in the x axis, the IgG group was injected during the 4 weeks with control isotype antibodies (due to inter-animal variability and smaller n per group, IgG1 and IgG4 groups were pooled), the anti-IL-15 group with anti-IL-15 antibody, and the IgG+anti-IL-15 group with control isotype antibody for 2 weeks then anti-IL-15 antibody for 2 weeks. Symbols represent individual xenografted mouse and groups are represented as violin plots showing group median and quartiles.

FIG. 6: Anti-IL-15 antibody does not affect numbers of dermal T (A: CD3+), NK (B: CD56+) and NKT (C: CD3+CD56+) cells in humanized atopic dermatitis model as described in Example 2 analyzed 4 weeks after injection of LPS-activated Th2-polarized PBMC in various antibody treatment groups by immunofluorescence. As indicated in the x axis, the IgG group was injected during the 4 weeks with control isotype antibodies (due to inter-animal variability and smaller n per group, IgG1 and IgG4 groups were pooled), the anti-IL-15 group with anti-IL-15 antibody, and the IgG+anti-IL-15 group with control isotype antibody for 2 weeks then anti-IL-15 antibody for 2 weeks. Symbols represent individual xenografted mouse and groups are represented as violin plots showing group median and quartiles.

FIG. 7: Anti-IL-15-induced decrease the expression of cytokines associated with atopic dermatitis in humanized atopic dermatitis model measured and analyzed in the dermis of human skin xenotransplants 4 weeks after injection of Th2-polarized PBMC in various antibody treatment groups as described in Example 2. A: IFN-γ; B: IL-4; C: IL-17A; D: IL-22). The number of cells expressing either IL-4, IL-17A, or IL-22 was calculated for each xenotransplant by adding the number of cells expressing each cytokine (E). As indicated in the x axis, the IgG group was injected during the 4 weeks with control isotype antibodies (due to inter-animal variability and smaller n per group, IgG1 and IgG4 groups were pooled), the anti-IL-15 group with anti-IL-15 antibody, and the IgG+anti-IL-15 group with control isotype antibody for 2 weeks then anti-IL-15 antibody for 2 weeks. Symbols represent individual xenografted mouse and groups are represented as violin plots showing group median and quartiles.

FIG. 8: Heatmap showing Log2FC of differentially regulated genes clustered by immune cell types (T helper cells, T cells, B cells, left) or Atopic Dermatitis-relevant genes (right) after therapeutic (IgG-CALY) or prophylactic (CALY) treatment with anti-IL-15 antibody in humanized atopic dermatitis mouse model, versus control isotype alone as described in Example 2. The lighter the shade, the higher the fold difference. The only upregulated gene is IL32, all other genes unchanged or downregulated. Stars indicate significantly regulated p value <0.05 (non-parametric Mann-Whitney test). All data are based on Nanostring transcriptomic analysis (10 xenotransplants from 3 separate human donors per group).

FIG. 9: Anti-IL-4R antibody and dexamethasone affect IL-15 expression differentially between the epidermis and dermis in humanized atopic dermatitis model as analyzed in human skin xenotransplants two weeks after injection of LPS-activated Th2-polarized PBMC in various treatment as described in Example 2. As indicated in the x axis, the IgG group was injected during the 4 weeks with control isotype antibodies (due to inter-animal variability and smaller n per group, IgG1 and IgG4 groups were pooled), the Dexamethasone group for the last 2 weeks of the study with topical dexamethasone, the anti-IL-4R group with anti-IL-4R antibody, and the IgG+anti-IL-4R group with control isotype antibody for 2 weeks then anti-IL-4R antibody for 2 weeks. In the epidermis, IL-15 expression was quantified as mean intensity signal, while in the dermis IL-15 expression was quantified as number of IL-15 positive cells per 100 dermal area units. Symbols represent individual xenografted mouse and groups are represented as violin plots showing group median and quartiles.

FIG. 10: Normalized gene expression values of IL-15 expression in skin biopsies between Healthy Volunteers and Atopic Dermatitis patient groups, at baseline (WO) or after 4 weeks (W4) or 16 weeks of Dupilumab treatment as described in Example 3. NL: non-lesional skin; L: lesional skin. Each symbol represents a separate individual, bars represent the mean value per group and error bars represent the 95% confidence interval. Statistical analyses were performed with Graph Pad Prism software. FIG. 1, 3, 9: A non-parametric Mann-Whitney test was used to compare the two experimental groups. (FIG. 1: ****p<0.0001; 4: **p<0.01,***p<0.001; 9: **p<0.01,***p<0.001). FIG. 2, 7: A one-way ANOVA Kruskal-Wallis test was used to compare the two groups with anti-IL-4R antibody or betamethasone to Th2-polarizing medium alone (FIG. 2: *p<0.05; FIG. 7: *p<0.05, **p<0.01). FIG. 4, 5, 6: A one-way ANOVA test with Dunnett's multiple comparison test was used to compare experiment groups (FIG. 4: *p<0.05; 5A: ****p<0.0001; 6B: *p<0.05; **p<0.01; 5C: *p<0.05, **p<0.01; 7: (ns: not significant).

FIG. 10: A one-way ANOVA test with Sidak's multiple comparison test was used to compare experiment groups ((*p<0.05; **p<0.01; ***p<0.001; ****p<0.0001, ns: not sigruficant).

### DETAILED DESCRIPTION OF THE INVENTION

The terms “interleukin 15”, “interleukin-15”, “IL-15”, designate herewith the interleukin 15 protein, also known as MGC9721, that is a 14 to 15 kDa pro-inflammatory cytokine that, in humans is encoded by the IL-15 gene whose sequence is disclosed under Hugo Gene Nomenclature Committee ID 5977. The immature form of IL-15 comprises 162 amino acids, where the first 29 amino acids constitute the signal peptide, and the amino acids 30 to 48 constitute the pro-peptide. The immature form of IL-15 is available under UniProtKB accession number P40933. The mature form of the IL-15 protein corresponds to amino acids Asn 49 to Ser 162, where the indicated positions correspond to the amino acid positions on the immature IL-15 amino acid sequence. The amino acid sequence of human mature IL-15 corresponds to the sequence disclosed in UniProtKB accession number P40933. The amino acid sequences of immature IL-15 from other species are available in the art and include, for instance, mouse IL-15 (UniProtKB accession number P48346, corresponding to mature IL-15 form), rat IL-15 (UniProtKB accession number P97604, corresponding to mature IL-15 form), Rhesus macaque IL-15 (UniProtKB accession NP_001038196, XP_001091166, XP_001091289 XP_001091416, corresponding to mature IL-15 form), Cynomolgus monkey IL-15 (predicted sequence from NCBI accession number XP_005556036.1, corresponding to mature IL-15 form) and dog IL-15 (UniProtKB accession A0A8C0NA66_CANLF corresponding to the full IL-15 sequence). The term “interleukin 15” also includes any variants or isoforms of interleukin 15 which are naturally expressed by cells. Of note, two alternatively spliced transcript variants of IL-15 have been reported. Although both isoforms produce the same mature protein, they differ in their cellular trafficking.

The term “IL-15 inhibitors” refers to agents able to inhibit interleukin 15. Inhibition of interleukin 15 can be assessed for example by measuring inhibition of IL-15 binding to signaling chains of the IL-15 receptor (Il-15Rβ and/or IL-15Rγ) or the IL-15-specific IL-15Rα chain, using techniques such as surface plasmon resonance or ELISA. Inhibition of interleukin 15 can be also assessed at the functional level for example by testing the inhibitory effect by measuring i) inhibition of signaling events downstream of IL-15 such as STAT5 phosphorylation that can be measured in tissue sections, whole cells or cells extracts using Western-blot, flow cytometry or immunofluorescence techniques; ii) inhibition of IL-15-dependent cell survival or activation or cytokine secretion or expression of certain genes in cellular assays, including cell lines or primary cells from animals, healthy humans or patients; iii) inhibition of IL-15 dependent mechanisms in vivo such as NK and other leukocyte homeostasis that can be measured by flow cytometry in the blood of animals, healthy humans or patients. Among those agents, some interleukin 15 inhibitors were or are being actively developed for the treatment of different diseases such as described in WO 2016/001275, WO 2015/089217, WO 2005/044303, WO 2018/119246, WO 2011/127324.

Interleukin 15 inhibitors can be small molecules inhibitors (e.g. low molecular weight organic compounds <900 Da), peptides, mAbs, chimeric proteins or fusion proteins, aptamers (including peptide aptamers, DNA and RNA aptamers), soluble receptors and such agents may also be acting by silencing, or down-regulating interleukin 15 expression.

The term “antibody” as referred to herein designates a polypeptide that binds an antigen. This includes whole antibodies and any antigen-binding fragments. The term “antibody” is used in its broadest sense and includes monoclonal antibodies, polyclonal antibodies, human antibodies, humanized antibodies, chimeric antibodies, bi-specific antibodies, multi-specific antibodies and further engineered antibodies as long as the characteristic properties of the invention are retained, in particular the ability of binding the target antigen (i.e. IL-15). Examples of antibodies are described herein. Bi- or multi-specific antibodies can be designed as described in Sawant et al., 2020, Int. J. Mol. Sci. 2020, 21, 7496. https://doi.org/10.3390/ijms21207496. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. The modifier “monoclonal” indicates the character of the antibody as being obtained from a substantially homogeneous population of antibodies, and is not to be construed as requiring production of the antibody by any particular method.

The term “chimeric antibody” generally refers to an antibody comprising a variable region from one source or species and at least a portion of a constant region derived from a different source or species, usually prepared by recombinant DNA techniques. A typical example of chimeric antibodies includes those comprising a mouse variable region and a human constant region. As defined herewith this term also includes an antibody comprising at least one of the CDRs of a first human antibody and at least a portion of a constant region of a second human antibody. It also includes an antibody comprising heavy chain CDR1, CDR2, and CDR3 of a first human antibody and light chain CDR1, CDR2, and CDR3 of a second human antibody.

The term “humanized antibody” designates antibodies from a non-human species having one or more complementarity determining regions (CDRs) from said non-human species and a framework region from a human immunoglobulin molecule. Humanized antibodies may optionally further comprise one or more framework residues derived from the non-human species from which the CDRs were derived.

The term “human antibody” or “fully human antibody” refers to antibodies in which the variable regions and the constant regions of both the heavy and the light chains are all of human origin, or substantially identical to sequences of human origin, but not necessarily from the same antibody.

The term “isolated antibody” refers to an antibody that has been separated from a component of its natural environment. For instance, an isolated antibody has been purified to greater than 95% or 99% purity as determined by methods in the art (see e.g. Flatman et al, 2007, J Chromatogr B Analyt Technol Biomed Life Sci, 848: 79-87) including electrophoretic (e.g. SDS-PAGE, isoelectric focusing, capillary electrophoresis) or chromatographic (e.g. ion exchange or reverse phase HPLC (high performance liquid chromatography) methods.

The term “variant” can apply to a polynucleotide and/or a polypeptide. For instance, a variant of a peptide or polypeptide, as referred to herein means a peptide or polypeptide substantially homologous to the referenced peptide sequence, but which has an amino acid sequence different from that of the referenced sequence because of one or more amino acid deletions, insertions and/or substitutions. Substantially homologous means a variant amino acid sequence which is identical to the referenced peptide sequence except for the deletion, insertion and/or substitution of a few amino acids, e.g. 1, 2, 3, 4, 5, or 6 amino acids. Substantially homologous means a variant amino acid sequence that is at least 80%, at least 85%, at least 90%, at least 91%, at least 92%, at least 93%, at least 94%, at least 95%, at least 96%, at least 97%, at least 98% or at least 99% identical to the referenced amino acid sequence. A variant nucleic acid sequence can be at least 80%, at least 85%, at least 90%, at least 91%, at least 92%, at least 93%, at least 94%, at least 95%, at least 96%, at least 97%, at least 98% or at least 99% identical to the referenced nucleic acid sequence. The identity of two amino acid sequences or of two nucleic acid sequences can be determined by visual inspection and/or mathematical calculation, or more easily by comparing sequence information using known computer program used for sequence comparison such as Clustal package version 1.83. A variant may comprise a sequence having at least one conservatively substituted amino acid, meaning that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions, for example, substitutions of entire regions having similar hydrophobicity characteristics, are well known (Kyte, et al, 1982, J. Mol. Biol., 157: 105-131). For example, a “conservative amino acid substitution” may involve a substitution of a native amino acid residue with a non-native residue such that there is little or no effect on the polarity or charge of the amino acid residue at that position. Alternatively, substitutions for one or more amino acids present in the original polypeptide are not conservative, which may generate a variant with modified properties compared to the antibody of reference. Desired amino acid substitutions (whether conservative or non-conservative) can be determined by those skilled in the art at the time such substitutions are desired. The term “variant” also includes a peptide or polypeptide substantially homologous to the referenced peptide sequence, but which has an amino acid sequence different from that of the referenced sequence because one or more amino acids have been chemically modified or substituted by amino acid analogs. This term also includes glycosylated polypeptides.

As used herewith the term “bind” or “binding” of an inhibitor to a target antigen means an at least temporary interaction or association of said inhibitor with, or to, said target antigen (e.g. IL-15) or with, or to, fragments of said target antigen comprising an epitope recognized by said inhibitor. As used herewith, an antibody binding IL-15 is also called an anti-IL-15 antibody.

The terms “selectively binds”, “specifically binds”, “specific for”, when applied to an antibody, indicate that the antibody preferentially recognizes and/or binds the target polypeptide or epitope, i.e. with a higher affinity than to any other antigen or epitope, i.e. the binding to the target polypeptide can be discriminated from non-specific binding to other antigens. The binding affinity of an antibody can be readily determined by one of ordinary skill in the art, for example, by equilibrium dialysis, equilibrium binding, surface plasmon resonance or spectroscopy (e.g. using a fluorescence assay). Especially, when using the surface plasmon resonance (SPR) technology, biomolecular binding events cause changes in the refractive index at a surface layer where one of the binding partner is immobilized, which are detected as changes in the surface plasmon resonance signal expressed as response units (RU). By measuring the real-time binding kinetics of an antibody to its target antigen, the SPR technology can determine how fast is the association between the antibody and its target (measured as kₐ or kₒₙ association constant), how strong is its association (measured as k_{d} or k_{off} dissociation constant). The affinity of an antibody for its target can be quantitatively measured by determining its equilibrium dissociation constant, K_{D}, defined as K_{D}=k_{d}/kₐ where kₐ is the association rate (kₒₙ) and k_{d} the dissociation rate (k_{off}) (Murphy, et al, 2006, Curr Protoc Protein Sci, Chapter 19: Unit 19.14). Comparison of affinity and/or binding properties between two antibodies can be established without actually determining the K_{D} value for each antibody, but based on a quantitative measurement of binding (e.g. by ELISA or FACS analysis) that is proportional to K_{D} or a qualitative measurement of affinity or an inference of affinity (e.g. in functional assay or in vitro or in vivo assay).

The term “blocking” or “neutralizing” activity of an inhibitor refers to its ability to inhibit its target's activity. The neutralizing activity of an inhibitor may be determined by in vitro assays or in vivo assays or functional assays. Applied to an inhibitor binding IL-15, this term refers to the inhibitor's ability to generally neutralize IL-15 activity, which can correspond for instance to the inhibition of the IL-15-induced proliferation and/or survival of activated T cells, natural killer cells, natural killer T cells and B lymphocytes or any other cell expressing the heterotrimeric IL-15Rαβγ or the heterodimeric IL-15Rβγ receptor (Finch, et al, 2011, Br J Pharmacol. 162:480-90), the IL-15-induced immunoglobulin synthesis by B lymphocytes stimulated by anti-IgM or CD40 ligand (Litinskiy et al, 2012, Nat Immunol., 3:822-9), the IL-15 induced activation of human neutrophils (Rathhé and Girard, 2004, J Leukoc Biol., 76:162-8), and the IL-15-induced production of proinflammatory cytokines from macrophages, dendritic cells or epithelial cells (Nanayakkara, et al, 2013, Am J Clin Nutr., 98:1123-35).

Unless otherwise constrained by the definition of the individual substituent, the term “substituted” refers to groups substituted with from 1 to 5 substituents selected from the group consisting of “C₁-C₆ alkyl,” “C₃-C₈-cycloalkyl,” “heterocycloalkyl,” “sulfonyl,” “sulphonamide”, “alkoxy,” “alkoxy carbonyl,” “halogen,” “carboxy,” “halomethyl”, cyano, hydroxy, nitro, and the like.

The term “pharmaceutically acceptable” refers to a carrier comprised of a material that is not biologically or otherwise undesirable.

The term “carrier” refers to any components present in a pharmaceutical formulation other than the active agent and thus includes diluents, binders, lubricants, disintegrants, fillers, coloring agents, wetting or emulsifying agents, pH buffering agents, preservatives and the like.

As used herein, “treatment” and “treating” and the like generally mean obtaining a desired pharmacological and physiological effect. The effect may be prophylactic in terms of preventing or partially preventing a disease, symptom or condition thereof and/or may be therapeutic in terms of a partial or complete cure of a disease, condition, symptom or adverse effect attributed to the disease. The term “treatment” as used herein covers any treatment of a disease in a mammal, particularly a human, and includes: (a) preventing the disease from occurring in a subject which may be predisposed to the disease but has not yet been diagnosed as having it for example based on familial history; (b) inhibiting the disease, i.e., arresting its development; or (c) relieving the disease, i.e., causing regression of the disease and/or its symptoms or conditions such as improvement or remediation of damage. For instance, treatment of celiac disease comprises preventing, decreasing or even eradicating the symptoms of the disease or disorder, for instance partial or total alleviation of atopic dermatitis symptoms such as dry skin, itching, scratching and the consequences of scratching (raw, sensitive, swollen skin), skin lesions including i) red to brownish-gray patches (especially on the hands, feet, ankles, wrists, neck, upper chest, eyelids, inside the bend of the elbows and knees, and in infants, the face and scalp); ii) small, raised bumps, which may leak fluid and crust over when scratched, and iii) thickened, cracked, scaly skin.

The term “subject” as used herein refers to mammals. For example, mammals contemplated by the present invention include human, primates, laboratory rodents, and pets such as dogs and cats and the like.

The term “efficacy” of a treatment or method according to the invention can be measured based on changes in the course of disease or condition in response to a use or a method according to the invention. For example, the efficacy of a treatment or method according to the invention can be measured by its impact on signs or symptoms of illness. A response is achieved when the patient experiences partial or total alleviation, or reduction of unwanted symptoms of illness.

The term “effective amount” as used herein refers to an amount of at least one agent according to the invention, or a pharmaceutical formulation thereof, that elicits a detectable reduction of the symptoms of the disease in a subject that is being administered said agent.

### IL-15 Inhibitors

IL-15 inhibitors according to the invention includes anti-IL antibodies, inhibitory peptides and small molecules.

According to one embodiment, IL-15 inhibitors can be small molecules such as those described in Quemener et al., 2017, J. Med. Chem. 2017, 60, 6249-6272 DOI: 10.1021/acs.jmedchem.7b00485, in particular compounds having the following structure:

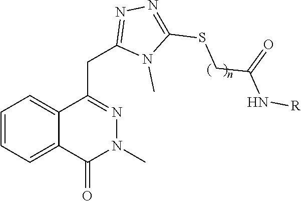

wherein R is an optionally substituted phenyl and n is an integer selected from 1 to 10, in particular a molecule having the following structure:

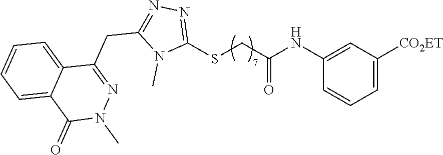

According to another embodiment, IL-15 inhibitors can be peptides such as IL-15 antagonists described in WO 2020/227019 and in WO 2015/089217 such as BNZ132 (aka BNZ-1, aka YT033, aka EQ-101) (SEQ ID NO: 2) (IKEFLQRFIHIVQSIINTS) and in US 2019/0070263 such as PKEFLERFVHLVQMFIHQSLS (SEQ ID NO: 3) or a fragment or variant of those sequences. Further peptidic IL-15 inhibitors are described in U.S. Pat. No. 7,736,638 (SEQ ID NO: 4) or a fragment or variant of those sequences.

According to another embodiment, IL-15 inhibitors can be anti-IL15 antibodies or antigen-binding fragments thereof. According to a particular aspect, anti-IL15 antibodies, or antigen-binding fragments thereof bind to IL-15, in particular human IL-15, or a fragment of IL-15.

Anti-IL15 antibodies according to the present invention generally exhibit a high specificity for human IL-15. However, depending on the degree of sequence identity between IL-15 homologs of different species, a given antibody or antigen-binding fragment may show cross-reactivity with IL-15 from at least one other species, e.g. primates (e.g. cynomolgus monkey, rhesus macaque, marmoset), mouse, rat, dog, and/or rabbit. For antibodies directed towards human IL-15, some level of cross-reactivity with other mammalian forms of IL-15 may be desirable in certain circumstances.

In a specific embodiment, the anti-IL15 antibodies according to the invention or fragments thereof bind preferentially to human IL-15.

In a still further embodiment, the antibodies according to the invention or antigen-binding fragments thereof show no cross-reactivity with rat IL-15 and/or mouse IL-15.

In one embodiment, the inhibitors according to the invention preferentially inhibit IL-15 and, optionally, additionally exhibit some inhibition to other proteins having homology with IL-15 such as IL-2, in particular human IL-2 or IL-21 (as disclosed in WO 2015/089217).

The ability of an antibody to block or neutralize the activity of its target protein can be evaluated by its potency as defined herewith, which is itself reflected, for instance, by the IC₅₀ value. Typically, the neutralizing activity of an antibody may be determined by in vitro assays, such as an assay for measuring the level of inhibition of IL-15-induced proliferation and/or survival of cell lines such as Kit 225 or M-07e cells, in the presence of said antibody, as described in WO 2016/001275.

In some embodiments, the antibodies, and antigen-binding fragments thereof, according to the invention have a IC₅₀ equal to or lower than 200 nM, in particular lower than 100 nM, in particular lower than 50 nM, lower than 30 nM, lower than 20 nM, more particularly lower than 10 nM, lower than 8 nM, lower than 7 nM, lower than 5 nM, lower than 4 nM, lower than 3 nM, lower than 2 nM, lower than 1 nM, lower than 0.5 nM, lower than 0.3 nM, lower than 0.2 nM, lower than 0.1 nM, lower than 0.05 nM, or lower than 0.03 nM, for inhibiting IL-15 activity such as IL-15 induced proliferation and/or survival of cell lines such as Kit 225 or M-07e cells as described in WO 2016/001275.

It is understood that any variant or fragment of an antibody described therein which is able to bind IL-15 and optionally neutralize IL-15 activity would be a suitable IL-15 inhibitor according to the invention. In a particular embodiment, such variant can show the same or even higher binding affinity for IL-15 and/or the same or even higher potency and/or the same or greater species-selectivity and/or the same or greater selectivity for IL-15, and/or the same or greater neutralizing efficacy, in comparison to the parental antibody or fragment from which said variant derives.

In a particular embodiment of the invention, the antibodies to IL-15, or antigen-binding fragments thereof which bind to IL-15, are monoclonal antibodies.

In a particular embodiment of the invention, the antibodies to IL-15, or antigen-binding fragments thereof which bind to IL-15, are bi- or multi-specific antibodies, in particular with bi-specific targeting IL-15 and IL-4R or IL-13 or IL-4.

In a further particular embodiment of the invention, the antibodies to IL-15 or antigen-binding fragments thereof which bind to IL-15, are humanized antibodies.

In a further particular embodiment of the invention, the antibodies to IL-15 or antigen-binding fragments thereof which bind to IL-15, are recombinant antibodies.

The antibodies to IL-15 or antigen-binding fragments thereof which bind to IL-15 and which are suitable for use according to the invention, can be characterized by their portion interacting with the target's protein, in particular by their variable region, which typically comprises a heavy chain variable region and a light chain variable region such as those described in WO 2016/001275.

According to a particular embodiment, anti-IL-15 inhibitor is an isolated antibody binding IL-15 or an antigen-binding fragment thereof comprising:
(1) a heavy chain variable region of SEQ ID NO: 5 or any variant thereof wherein said variant has the amino acid sequence of SEQ ID NO: 5 except that 1, 2, 3 or 4 amino acids are substituted by a different amino acid, wherein substitutions are selected among:
   (i) arginine (R) at position H3 (VH RH3) substituted by glutamine (Q), methionine (M) at position H5 (VH MH5) substituted by valine (V), alanine (A) at position H6 (VH AH6) substituted by glutamic acid (E), alanine (A) at position H49 (VH AH49) substituted by serine (S), within the heavy chain variable framework region,
   (ii) aspartic acid (D) at position H61 (VH DH61) substituted by glutamic acid (E), serine (S) at position H62 (VH SH62) substituted by threonine (T), within the heavy chain CDR2, and
   (iii) methionine (M) at position H98 (VH MH98) substituted by leucine (L), phenylalanine (F), isoleucine (I), or alanine (A), tryptophan (W) at position H100C (VH WH100C) substituted by tyrosine (Y), phenylalanine (F) or alanine (A), methionine (M) at position H100E (VH MH100E) substituted by leucine (L), phenylalanine (F) or isoleucine (I), within the heavy chain CDR3;
   and
(2) a light chain variable region of SEQ ID NO: 9.

According to another particular embodiment, anti-IL-15 inhibitor is an isolated antibody binding IL-15 or an antigen-binding fragment thereof comprising:
(1) a heavy chain variable region of SEQ ID NO: 6 or any variant thereof wherein said variant has the amino acid sequence of SEQ ID NO: 6 except that 1, 2, 3 or 4 amino acids are substituted by a different amino acid and
(2) a light chain variable region of SEQ ID NO: 9.

According to another particular embodiment, anti-IL-15 inhibitor is an isolated antibody or antigen-binding fragment thereof according to claim 1, comprising:
(1) a heavy chain variable region of amino acid sequence of SEQ ID NO: 5 having substitutions in said sequence of SEQ ID NO: 5 selected among:
   (i) VH RH3 is substituted by glutamine (Q), VH MH5 is substituted by valine (V), VH AH6 is substituted by glutamic acid (E),
   (ii) VH SH62 is substituted by threonine (T), and
   (iii) VH WH100C is substituted by tyrosine (Y);
and
(2) a light chain variable region of amino acid sequence of SEQ ID NO: 9.

According to another particular embodiment, anti-IL-15 inhibitor is an isolated antibody binding IL-15 or an antigen-binding fragment thereof comprising:
(1) a heavy chain variable region selected from: SEQ ID NO: 5, SEQ ID NO: 6, SEQ ID NO: 7 and SEQ ID NO: 8, and
(2) a light chain variable region of SEQ ID NO: 9.

According to another particular embodiment, anti-IL-15 inhibitor is an isolated antibody binding IL-15 or an antigen-binding fragment thereof comprising a heavy chain variable region of SEQ ID NO: 6 and a light chain variable region of SEQ ID NO: 9.

An interleukin-15 inhibitor for use according to claim 1 or 6, wherein said interleukin-15 inhibitor selected from an anti-IL-15 antibody comprising:
a heavy chain of SEQ ID NO: 10 and a light chain of SEQ ID NO: 11 or a variant or fragment thereof; or
a variable heavy chain region of SEQ ID NO: 12 or a variant or fragment thereof and a variable light chain region of SEQ ID NO: 13 or a variant or fragment thereof; or
a variable heavy chain region of SEQ ID NO: 14 or a variant or fragment thereof and a variable light chain region of SEQ ID NO: 15 or a variant or fragment thereof; or
a heavy chain of SEQ ID NO: 16 and a light chain of SEQ ID NO: 17 or a variant a variant or fragment thereof, or
a heavy chain of SEQ ID NO: 18 and a light chain of SEQ ID NO: 17 or a variant a variant or fragment thereof, or
a heavy chain of SEQ ID NO: 19 and a light chain of SEQ ID NO: 17 or a variant a variant or fragment thereof.

According to another further particular embodiment, anti-IL-15 inhibitor is an isolated antibody binding IL-15 or an antigen-binding fragment thereof comprising a heavy chain of SEQ ID NO: 16 and a light chain of SEQ ID NO: 17.

According to another further particular embodiment, anti-IL-15 inhibitor is an isolated antibody binding IL-15 or an antigen-binding fragment thereof comprising a heavy chain of SEQ ID NO: 18 and a light chain of SEQ ID NO: 17.

According to another further particular embodiment, anti-IL-15 inhibitor is an isolated antibody binding IL-15 or an antigen-binding fragment thereof comprising a heavy chain of SEQ ID NO: 19 and a light chain of SEQ ID NO: 17.

According to another particular embodiment, anti-IL-15 inhibitor is an isolated antibody binding IL-15 or an antigen-binding fragment thereof comprising a sequence 146B7 (aka AMG174) as described in WO 2005/044303 or in WO 03/017935 being an IL-15 antagonist is a monoclonal human IgG1 anti-IL-15 antibody having heavy chain and kappa light chain regions which comprise the amino acid sequences of SEQ ID NO: 10 and SEQ ID NO: 11.

According to another particular embodiment, anti-IL-15 inhibitor is an isolated antibody binding IL-15 as described in WO 2018/119246, or an antigen-binding fragment thereof comprising a variable heavy chain sequence of SEQ ID NO 12 and a variable light chain sequence of SEQ ID NO 13.

According to another particular embodiment, anti-IL-15 inhibitor is an isolated antibody binding IL-15 as described in WO 2011/127324 or an antigen-binding fragment thereof, in particular huABC2 (anti-IL-15Rβ antibody) comprising a variable heavy chain sequence of SEQ ID NO 14 and a variable light chain sequence of SEQ ID NO 15.

According to another particular embodiment, anti-IL-15 inhibitor is an isolated antibody binding IL-15 as described in WO 2009/002562 or an antigen-binding fragment thereof, or in WO 2017/217985 & US 2018/0002417 or in WO 2005/044303 and WO 03/017935 such as 146B7 or an antigen-binding fragment thereof.

Specific examples of the antibodies according to the invention include those listed in Table 1.

TABLE 1

| | Heavy chain | Light chain |
|---|---|---|
| Antibody name | SEQ ID NO: | SEQ ID NO: |
| hu-BE29-2 | 16 | 17 |
| hu-BE29-10 | 18 | 17 |
| hu-BE29-24 | 19 | 17 |

### Conjugates Comprising an Auxiliary Molecule

In another aspect of the invention, the isolated antibodies or antigen-binding fragments thereof according to the invention are optionally conjugated to an accessory molecule, and are then also referred to herein as “conjugated antibodies or “conjugated antibody fragments”.

The conjugated antibodies and conjugated antibody fragments according to the invention can target anti-IL-15 inhibitor in vivo to a site of disease (e.g. a site of inflammation) such that the anti-IL-15 inhibitor can have a preferential concentration and therapeutic effect on the site of disease, and less side effects elsewhere in the body.

The accessory molecule may be conjugated to the antibody or antibody fragment directly or via a spacer of suitable length for instance as described in Kellogg et al. (2011, Bioconjug Chem, 22: 717-27).

In another embodiment, the accessory molecule comprises an antigen-binding fragment of an antibody, which, when conjugated to the antibody or antibody fragment according to the invention, form a bispecific antibody. In particular, said bispecific antibody may be directed to two different epitopes of IL-15 (hence defining a biparatopic antibody) or with bi-specific moieties targeting IL-15 and IL-4R or IL-13 or IL-4.

### Compositions

The IL-15 inhibitors are provided in the form of a pharmaceutically acceptable composition. Pharmaceutical compositions can contain one or more IL-15 inhibitors, in particular one or more antibodies binding IL-15 or antigen-binding fragments thereof in any form described herein.

Compositions of this invention may further comprise one or more pharmaceutically acceptable additional ingredient(s) such as alum, stabilizers, antimicrobial agents, buffers, coloring agents, flavoring agents, adjuvants, and the like.

IL-15 inhibitors together with a conventionally employed adjuvant, carrier, diluent or excipient may be placed into the form of pharmaceutical compositions and unit dosages thereof, and in such form may be employed as solids, such as tablets or filled capsules, freeze-dried forms, or liquids such as solutions, suspensions, emulsions, elixirs, or capsules filled with the same, all for oral use, or in the form of sterile injectable solutions for parenteral (including subcutaneous) use, in particular formulated in pre-filled syringes or auto-injectors. Such pharmaceutical compositions and unit dosage forms thereof may comprise ingredients in conventional proportions, with or without additional active compounds or principles, and such unit dosage forms may contain any suitable effective amount of the active ingredient commensurate with the intended dosage range to be employed.

Compositions may be liquid formulations including, but not limited to, aqueous or oily suspensions, solutions, emulsions, syrups, and elixirs. Liquid forms suitable for oral administration may include a suitable aqueous or non-aqueous vehicle with buffers, suspending and dispensing agents, colorants, flavors and the like. The compositions may also be formulated as a dry product for reconstitution with water or other suitable vehicle before use. Such liquid preparations may contain additives including, but not limited to, suspending agents, emulsifying agents, non-aqueous vehicles and preservatives. Suspending agent include, but are not limited to, sorbitol syrup, methylcellulose, glucose/sugar syrup, gelatin, hydroxyethylcellulose, carboxymethylcellulose, aluminium stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid. Further materials as well as processing techniques and the like are set out in Remington: The Science & Practice of Pharmacy, 23^{rd} Edition, 2020, Ed. Adeboye Adejare, Academic Press, which is incorporated herein by reference, which is incorporated herein by reference.

Solid compositions of this invention may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers include, but are not limited to, lactose, sugar, microcrystalline cellulose, maize starch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate. Tablets may be coated according to methods well known in the art.

Injectable compositions are typically based upon injectable sterile saline or phosphate-buffered saline or other injectable carriers known in the art.

Compositions may also be formulated as transdermal formulations comprising aqueous or non-aqueous vehicles including, but not limited to, creams, ointments, lotions, pastes, medicated plaster, patch, or membrane.

Compositions may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

Compositions may also be formulated as a depot preparation, which may be administered by implantation or by intramuscular injection. The compositions may be formulated with suitable polymeric or hydrophobic materials (as an emulsion in an acceptable oil, for example), ion exchange resins, or as sparingly soluble derivatives (as a sparingly soluble salt, for example).

The compounds can also be administered in sustained release forms or from sustained release drug delivery systems. A description of representative sustained release materials can also be found in the incorporated materials in Remington's Pharmaceutical Sciences. Injectable formulations are particularly appropriate for administering the compounds or compositions according to the invention.

### Combination

According to the invention, IL-15 inhibitor according to the invention can be administered alone or in combination with a co-agent useful in the prevention and/or treatment of atopic dermatitis, for example anti-IL-4R such as dupilumab, an anti-IL-13 such as tralokinumab, a JAK inhibitor such as abrocitinib, upadacitinib (oral) or ruxolitinib (topical) or a corticoid such as dexamethasone.

The invention encompasses use of an IL-15 inhibitor wherein the IL-15 inhibitor is to be administered to a subject prior to, simultaneously or sequentially with other therapeutic regimens or co-agents useful in the prevention and/or treatment of atopic dermatitis, in a therapeutically effective amount. The IL-15 inhibitor according to the invention that are administered simultaneously with said co-agents can be administered in the same or different compositions and in the same or different routes of administration.

In a particular embodiment, an IL-15 inhibitor, in particular an antibody for IL-15 or an antigen-binding fragment thereof according to the invention can be administered in combination with an anti-IL-4R antibodies such as dupilumab.

In a particular embodiment, an IL-15 inhibitor, in particular an antibody for IL-15 or an antigen-binding fragment thereof according to the invention can be administered in combination with an anti-IL-13 such as tralokinumab.

In a particular embodiment, an IL-15 inhibitor, in particular an antibody for IL-15 or an antigen-binding fragment thereof according to the invention can be administered in combination with one or more corticosteroid such as dexamethasone.

### Mode of Administration

Compositions of this invention may be administered in any manner including, but not limited to, orally, parenterally, sublingually, transdermally, transmucosally, topically, or combinations thereof.

Parenteral administration includes, but is not limited to, intravenous, subcutaneous, and intramuscular. The compositions of this invention may also be administered in the form of an implant, which allows slow release of the compositions as well as a slow controlled i.v. infusion.

In a particular embodiment, an IL-15 inhibitor is administered systemically or locally for example by topical application.

In a particular embodiment, an IL-15 inhibitor, in particular an antibody for IL-15 or antigen-binding fragment thereof is administered by subcutaneous or intravenous route.

The dosage administered, as single or multiple doses, to an individual will vary depending upon a variety of factors, including pharmacokinetic properties, subject conditions and characteristics (sex, age, body weight, health, size), extent of symptoms, concurrent treatments, frequency of treatment and the effect desired.

According to a particular embodiment, the inhibitor is administered from once a week to once a month.

Typically, therapeutically effective amounts of a pharmaceutically active antibody range from 0.1 mg/kg up to 50 mg/kg body weight dose.

### Patients

In an embodiment, subjects according to the invention are suffering from atopic dermatitis.

According to a particular embodiment, subjects according to the invention present high systemic or local levels of IL-15, or biomarkers of IL-15 pathway activation such as for example described in Karlen and Simon, 2020, Int. Arch. Allergy Immunol., 181, 417-421 and Kurowska et al., 2020, J. Clin. Med), 1555.

In another embodiment, subjects according to the invention are suffering from atopic dermatitis and are not responding to or cannot use anti-IL-4R, an anti-IL-13 and/or costicosteroid treatment. Subjects non responding to anti-IL-4R, an anti-IL-13 and/or corticosteroid treatments are defined by lack of satisfying response after treatments with anti-IL-4R, an anti-IL-13 and/or corticosteroid.

### Uses and Methods According to the Invention

In a particular embodiment is provided a method of preventing and/or treating atopic dermatitis, comprising administering a therapeutically effective amount of an IL-15 inhibitor, in particular an antibody for IL-15 or antigen-binding fragment thereof, to a subject in need thereof.

According to a particular embodiment, the interleukin-15 inhibitor is administered in the form of a formulation according to the invention.

References cited herein are hereby incorporated by reference in their entirety.

The invention having been described, the following examples are presented by way of illustration, and not limitation.

### EXAMPLES

### Example 1: IL-15 and IL-15Ra Expression in an Ex-Vivo Human Model of Atopic Dermatitis

The effects of an anti-IL-15 antibody (hu-BE29-2 comprising a heavy chain of SEQ ID NO:16 and a light chain of SEQ ID NO:17 as described in WO 2016/001275) in atopic dermatitis was investigated in the following experimental model.

The experimental setup uses healthy skin punch biopsies which are induced to display an atopic dermatitis phenotype using a cocktail which activates the Th2 pathway (https://www.reprocell.com/drug-efficacy-safety-adme/assay-catalog/skin-culture-induced-atopic-dermatitis-model-systemic-topical) as follows:

Up to 36×3 mm full thickness skin biopsies from three different healthy donors were obtained, as resection from surgical procedures as described below. On Day -1, skin biopsies are prepared, randomized and fully submerged in a minimum of 1 mL of basic culture medium overnight in a cell incubator. Following the equilibration period, biopsies are transferred to separate culture wells of a 12-well plate, at Day 0. Then a hole is cut in a Transwell™ filter using a pipette tip or forceps and each biopsy is inserted into the hole of one filter. The filter containing the biopsy is placed into one of the wells of the 12-well culture plate containing 1 mL of the appropriate culture medium with the epidermis facing upwards at the air-liquid interface. One mL of culture medium with or without a Th2-polarizing culture medium (Cousins et al., 2002, J. Immunol 169, 2498-2506) and test articles is added to each well at Day 0 and replaced at Day 1. The test compounds used in that study were an anti-human TL-4R antibody which sequence is identical to that of dupilumab (https://www.genome.jp/dbget-bin/www_bget?dr:D10354) and produced by transient transfection in Chinese Ovary Cells (Evitria AG, Zurich, Switzerland) and bethamethasone (Merck Life Sciences, Gillingham, Unite Kingdom), at a final concentration of 50 μg/mL, or betamethasone at a final concentration of 10 μM.

At approximately 48 h post application of Th2 stimulation cocktail and test compounds referenced above (Day 2), biopsies are harvested stored in RNAlater for a minimum of 24 h at 2-8° C. Following this, —RNAlater is removed, and biopsies snap frozen in liquid nitrogen and stored at 80° C. Tissue samples stabilized in RNAlater solution are processed for RNA extraction and isolation. RNA is quantified, and quality checked using a Qubit system. Once quality check is completed, RNA samples are submitted to RNAseq analysis.

### Skin and PBMC Human Donors

Healthy (control) abdominal human skin was obtained from three healthy volunteers (39-52 years-old) who underwent elective surgery and did not have a history of atopic disease. PBMCs were collected from 20 mL of venous blood from the same donors. The study was approved by the Rambam Health Care Campus Institutional Helsinki Committee (0182-14-RMB).

### PBMCs Stimulation and Characterization

Autologous PBMCs were isolated as described previously (Keren et al., 2018, J. Allergy Clin. Immunol., 142, 305-308.e6. https://doi.org/10.10161j.jaci.2018.02.015).

After isolation, they were cultured in the presence of IL-2 (10 U/mL), IL-4 (200 U/mL), and LPS (1 g/mL; Sigma, St. Louis, MO) (“Th2-polarized”) to induce a Th2 phenotype.

In the pilot experiment where induction of IL-15 expression was assessed, control PBMC were cultured in the presence of IL-2 and IL-4 but without LPS activation.

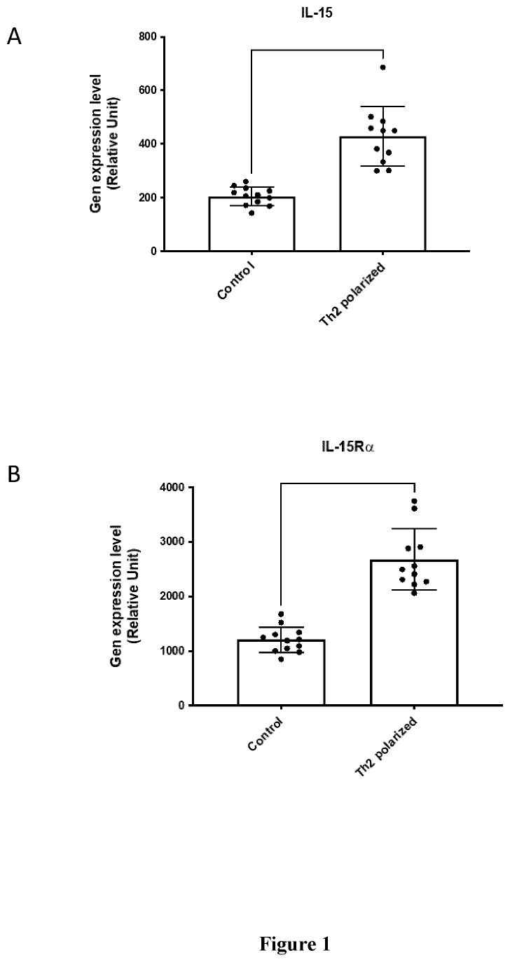

It was observed that Th2 culture conditions, known to induce genes similar to what found in atopic dermatitis lesions, are able to induce IL-15 gene expression, as well as IL-15Ra expression, in human skin explants in vitro as shown in FIG. 1A & B.

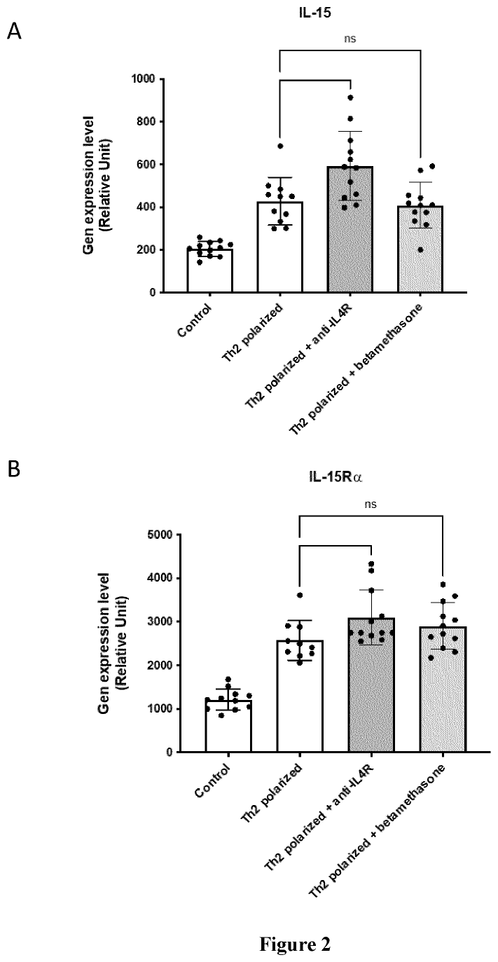

It was also observed that induced IL-15 and IL-15Ra expression is not decreased by treatment with an anti-IL-4R antibody or corticoids in ex vivo a human atopic dermatitis model as shown in FIG. 2A & B.

### Example 2: Anti-IL-15 Antibody is Effective in a Humanized Mouse Model of Atopic Dermatitis

The human atopic dermatitis model, however, does not well represent the human disease. Similarly, existing mouse experimental models have limited value, since they only represent human disease to a limited extent and rely on mouse skin and immune system components that differ from their human counterparts (Gilhar and Paus, 2021, Exp. Dermatol., 30, 319-336. https://doi.org/10.1111/exd.14270). Humanized mouse models offer a possibility to study human cell function in vivo, and in dermatology humanized models of psoriasis have proven to be, although complex and costly, most representative of the human disease. A “humanized” model of atopic dermatitis as described in Gilhar and Paus, 2021, supra was used. “Humanized” mice are defined here as immunodeficient (SCID/beige) mice transplanted with normal human skin from healthy donors as well as activated T helper (Th)2-polarized peripheral mononuclear cells (PBMC) from the same donor, to recapitulate clinical and histological features of atopic dermatitis at the level of the skin graft.

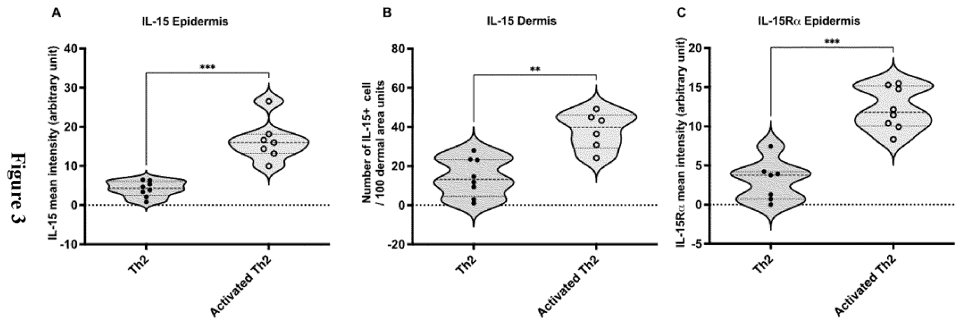

A first objective of the study was to assess if IL-15 was induced during the development of this atopic dermatitis model (pilot experiment). As can be seen on FIG. 3, it was demonstrated that both IL-15 and IL-15Rα expression was induced in this model. While IL-15Rα was mostly expressed by epidermal cells, IL-15 was expressed in by epidermal cells but also by dermal cells, presumably by cells of the monocyte and dendritic lineage.

This analysis validated the presence of the IL-15 target in the humanized atopic dermatitis mouse model, and therefore its suitability to test compounds that neutralize IL-15.

The secondary objective of the study was to determine whether an inhibitor of interleukin 15 as an anti-interleukin-15 antibody, given at different regimen, could be effective in this humanized model of atopic dermatitis.

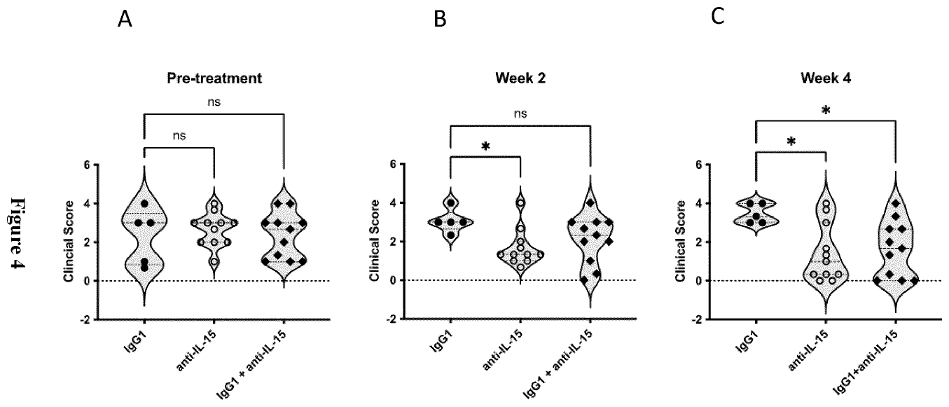

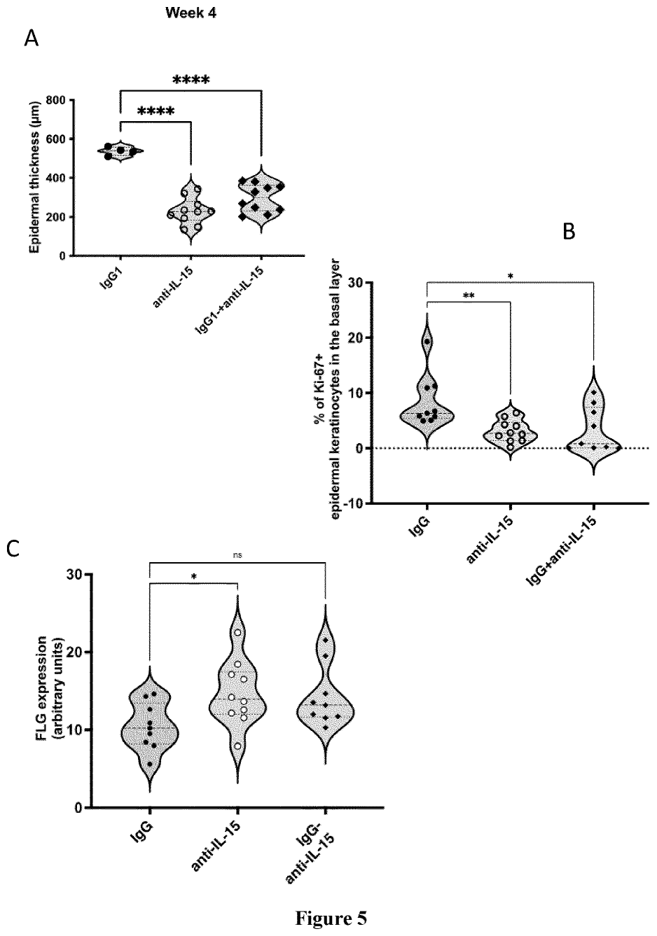

When administering an anti-IL-15 antibody during the 4 weeks of onset of the atopic dermatitis lesions, or only during the last 2 weeks, a significant effect on macroscopic skin lesions was observed as shown on FIG. 4 represented by clinical score based on the measuring of erythema and skin integrity as described below. Further, there was a significant effect of anti-IL-15 treatment on the epidermis properties (FIG. 5) such as epidermal thickness (A), epidermal basal cell proliferation (B), and expression of filaggrin (C) (a molecule related to skin barrier integrity, often mutated in human atopic dermatitis patients (O'Regan et al., 2008, J. Allergy Cin. Immunol., 122, 689-693. https://doi.org/10.1016/j.jaci.2008.08.002), all features known to be characteristics of human atopic dermatitis.

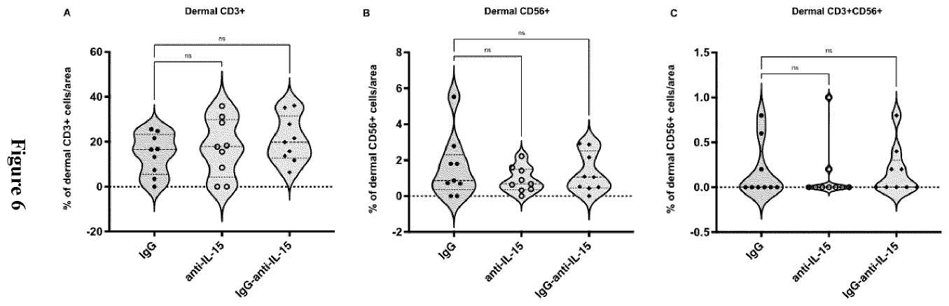

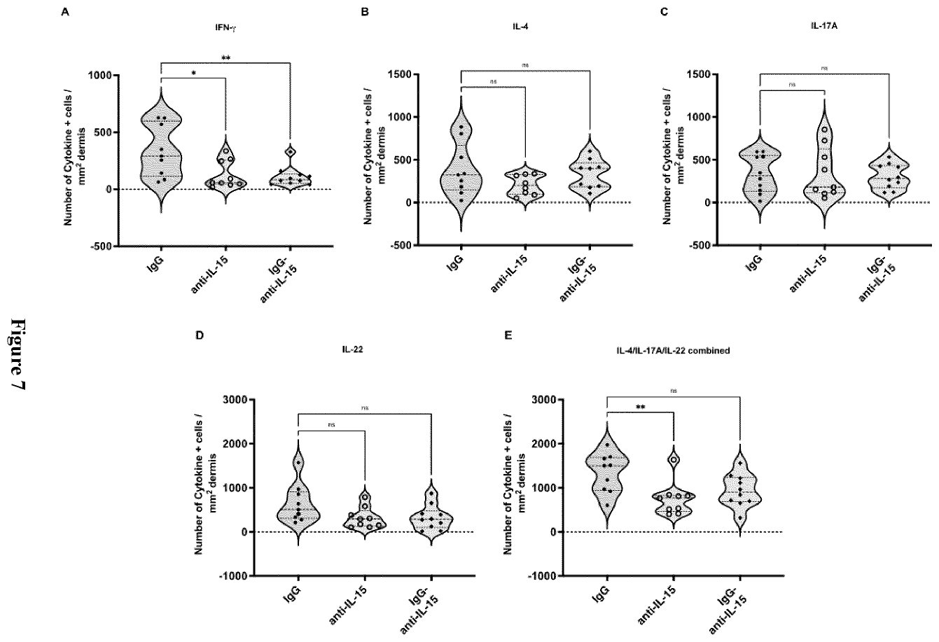

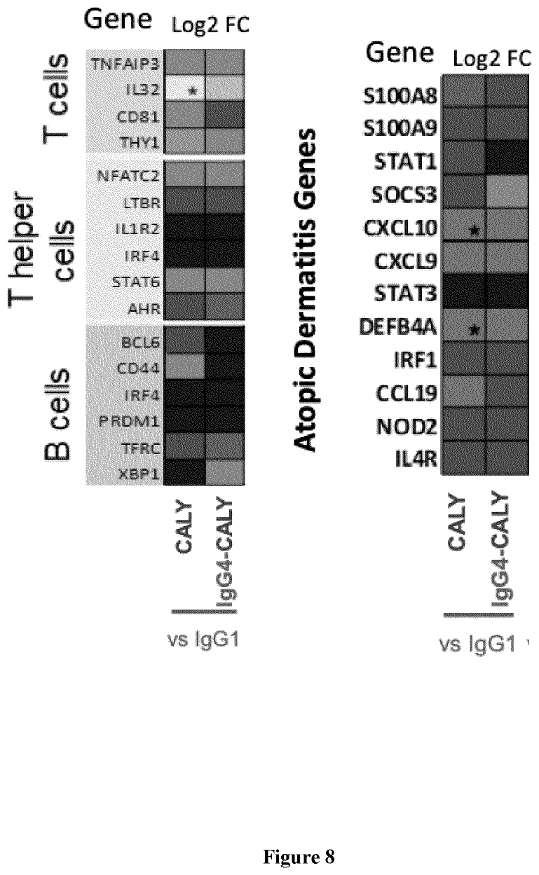

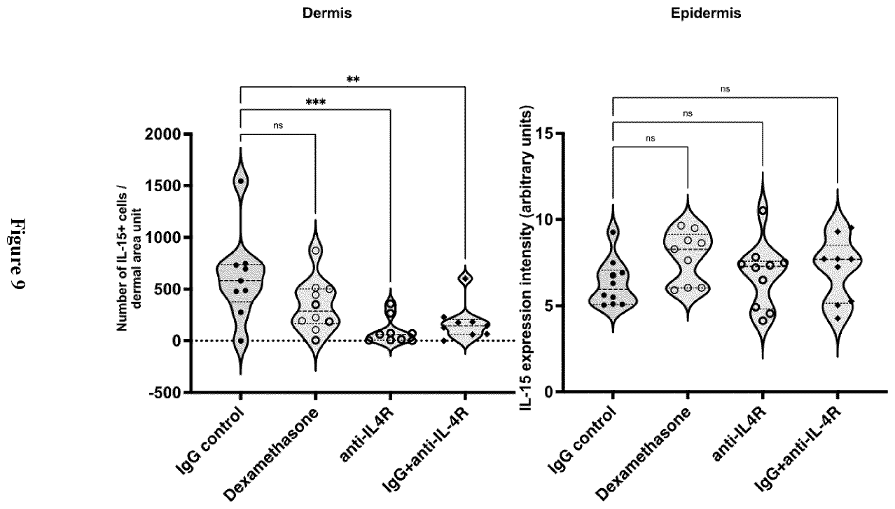

A third objective was to analyze in treated mice the effect of the anti-IL-15 antibody on key read-outs and biomarkers known to be associated with atopic dermatitis in humans. There was also an effect of anti-IL-15 therapy on cytokines potentially secreted by T cells and known to be induced in human atopic dermatitis (IFN-γ, IL-4, IL-17, and IL-22) (FIG. 7). On the other hand, anti-IL-15 therapy did not sensibly affect the number of T, NK and NKT cells present in the dermis of the human skin grafts (FIG. 6), those cells being thought to be responsible for IFN-γ, IL-4, IL-17, and IL-22 secretion. That suggests that either anti-IL-15 was only affecting the function of those T, NK and NKT cells, or that it was only affecting numbers and/or functions of a small subset of cells that could not be delineated with the techniques used herein. When looking at immune gene expression analysis as described below, it was observed that hu-BE29-2 (also called CALY-002) treatment induced a decreased expression of many immune genes related to T cells, T helper Cells and B cells as well as genes known to be associated with Atopic Dermatitis including S100A9, DEFB4, CXCL10 (FIG. 8). Overall, these data suggest that blocking the IL-15 pathway could be an efficient novel therapy for atopic dermatitis. Because both anti-IL-4R therapeutic antibody and corticoids are approved treatments for atopic dermatitis (Strowd et al., 2017, The Lancet, 39 (10086), 2265-2266), it was investigated whether those treatments could affect IL-15 expression in the atopic dermatitis models analyzed. Interestingly, in the ex vivo human explant model of Example 1, neither anti-IL-4R antibody nor corticoids could decrease IL-15 (FIG. 9), while both treatments could significantly decrease many other genes associated with atopic dermatitis. The profile was the same for IL-15Ra expression and in fact the expression of IL-15 and IL-15Ra was even slightly increased by anti-IL-4R treatment. In addition, in the humanized atopic dermatitis mouse model of the present Example, anti-IL-4R only decreased IL-15 expression in the dermis but not in the epidermis. Corticoids showed a trend, but not statistical significance, to a decrease expression of IL-15 limited to dermis. These results suggest that the IL-15 pathway is not fully inhibited by treatment with anti-IL-4R antibodies or corticoids and that therefore an anti-IL-15 therapy might be advantageously administered to patients not responding to anti-IL-4R or corticoids, or in combination with those or other treatments.

### Animals

A total of 60 female C.B-17/IcrHsd-scid-bg (beige-SCID, Harlan Laboratories Ltd., Jerusalem, Israel) mice, between 2 and 3 months of age were used in the therapy experiment. Mice were housed under pathogen-free conditions, compliant with institutional guidelines.

### Skin and PBMC Human Donors

Healthy abdominal human skin was obtained from healthy female volunteers who underwent elective surgery and did not have a history of atopic disease. PBMCs were collected from 20 mL of venous blood from the same donor. The study was approved by the Rambam Health Care Campus Institutional Helsinki Committee (0182-14-RMB).

### Humanized Mouse Atopic Dermatitis Model

Split-thickness skin xenotransplantation was performed as described (Keren et al., 2018, supra). Briefly, human skin samples (1 cm2, 0.4 mm thickness) from three different donors were transplanted onto mice (one human xenotransplant per mouse). One month after transplantation, the skin was injected intradermally with 1×10⁷ Th2-polarized PBMCs. Before injection, the viability of cells was determined by Trypan Blue staining.

In Vivo Treatment with Anti-IL-15 Antibody and Controls

Two treatments regimen with an anti-IL5 antibody hu-BE29-2 of SEQ ID NO:16 and SEQ ID NO:17) as described in WO 2016/001275 or controls were considered. Mice (10 per group) were intravenously injected with 200 μg/mouse anti-IL-15 antibody (manufactured by Calypso Biotech S.A., Switzerland) three times per week for 4 weeks following PBMC injection, or first with a matching dose of IgG1 control isotype antibody for 2 weeks and then with the anti-IL-15 antibody for 2 weeks. Other groups of mice (10 per group) were similarly injected with 200 μg/mouse anti-IL-4 receptor (IL-4R) antibody which sequence is identical to that of dupilumab (https://www.genome.jp/dbget-bin/www_bget?dr:D10354) and produced by transient transfection in Chinese Ovary Cells (Evitria AG, Zurich, Switzerland) for 4 weeks or first with IgG4 control isotype antibody for 2 weeks followed by anti-IL-4R antibody for 2 weeks. Negative controls (5 mice per group) were injected for 4 weeks with a matching dose of either IgG1 or IgG4 control antibodies (Evitria AG, Zurich, Switzerland). As a positive control, dexamethasone was locally applied three times per week starting from the third week after PBMC injection to a group of 10 mice. Macroscopic images of the transplanted skin were taken at Day 0 and Weeks 1, 2, 3 and 4 after PBMC injection. At the end of the 4 weeks of treatment, each xenografted skin explant was collected, cut in half and either embedded in Tissue-Tek® O.C.T. and stored frozen, or fixed in formalin, embedded in paraffin and stored at room temperature until further evaluations.

### Clinical Macroscopic Evaluation

To measure the severity of the atopic dermatitis-like phenotype in the xenotransplant skin, measurement usually applied to the clinic were adapted to the analysis of the collected macroscopic images, to grade the signs of erythema and skin integrity (excoriation/scaling/lichenification) on a discrete scale (0=absent, 1=moderate, and 2=severe) at Day 0 and Weeks 1, 2, 3 and 4 after PBMC injection. The total score (erythema score+skin integrity score) was calculated for each mouse. The scoring was performed blinded and independently by three operators.

### Hematoxylin and Eosin (H&E) Staining and Epidermal Thickness Measurement

Routine H&E staining was conducted on 7 μm-thick cryosections. The mean human epidermal thickness of each xenotransplant was determined on those H&E-stained sections by averaging the measured distance between the outermost surface of the epidermis excluding the stratum corneum and the dermo-epidermal junction at 10 randomly chosen points through the entire length of each examined section, with the aid of a digital caliper and ImajeJ software. The measurements, performed in a blinded manner, were then averaged within each experimental group.

### IL-15 and IL-15Rα Expression

For testing the expression of IL-15 and IL15-Rα in the xenografts, OCT (Optimal Temperature Cutting compound)-embedded skin xenotransplants were sectioned (7 μm thickness) with a Leica cryostat. Tissue cryosections were fixed in acetone, and pre-incubated with 10% of goat serum in PBS. The sections were then incubated with the corresponding primary antibody (anti-IL-15, Ab55276, Abcam, 1:100; anti-IL-15RA, Ab91270, Abcam, 1:100) at 4° C. overnight. Secondary antibody incubation was performed at RT for 45 min (Goat-anti-Mouse-IgG-Alexa546, Invitrogen, 1:500). Counterstaining with DAPI (1 μg/ml) was performed to visualize nuclei.

### Immunohistochemistry for Atopic Dermatitis Markers.

For measuring filaggrin (FLG) expression, cryopreserved xenograft skin sections were stained o.n. at 4° C. with a rabbit anti-filaggrin polyclonal antibody (Biolegend, 1:500-1:250) followed by species-specific fluorescent conjugated secondary antibody and DAPI counterstain to visualize the nuclei. Filaggrin expression (fluorescence intensity) was measured in 1-3 randomly selected epidermal areas of 3-6 cryosections for each xenograft with a fluorescence microscope at 10× magnification. A region of interest (ROI) including the human epidermal stratum corneum was manually selected on each image, and the mean FLG expression value within a ROI was calculated with Image J software. For each xenograft, mean filaggrin expression level was calculated.

T, NK and NKT cell infiltration was assessed by analyzing the expression of CD3 (T-cell marker) and CD56 (NK cell marker). Cryopreserved xenograft skin sections were double stained with a mouse anti-human CD56 antibody (Dako, 1:100, overnight at 4° C.) followed by tyramide signal amplification (TSA, enabling the detection of low-abundance targets) and with a mouse anti-CD3 monoclonal antibody Alexa Fluor 647 pre-conjugated (Invitrogen, 1:50, room temperature 1 h). The number of DAPI+, CD3+, CD56+, and CD3+ or CD56+ positive cells was calculated in 3-5 randomly selected 100 μm2 dermal areas in one cryosection for each xenografted skin. The mean values were calculated for each skin sample and expressed as percentage of DAPI+ cells/area.

Epidermal proliferation was analyzed by looking at the expression of the Ki67 proliferation marker. FFPE xenograft skin sections were deparaffinized and rehydrated. After heat induced antigen retrieval in sodium citrate buffer and blocking with normal goat serum, sections stained overnight at 4° C. with a mouse anti-Ki-67 antibody (M7240, Dako, 1:10) followed by anti-mouse fluorescent conjugated goat secondary antibody. The sections were briefly treated with DAPI to visualize the nuclei. For each xenografted skin, 2 sections were imaged with a fluorescence microscope at 20× magnification. The innermost layers of the epidermis were selected as the region of interest (ROI) and Ki-67+ cells were counted in the ROIs. To determine the total number of cells, DAPI-stained nuclei were counted with the thresholding automatic method coded in the ImageJ image analysis software.

IL-4 expression was analyzed on FFPE xenograft skin sections that were first deparaffinized and rehydrated. After heat induced antigen retrieval in citrate buffer and blocking with normal goat serum, sections were stained overnight at 4° C. with a rabbit anti-IL-4 antibody (Ab9622, Abcam, 1:100) followed by species-specific biotinylated goat secondary antibody. IL-4+ cells were determined with streptavidin-HRP targeting the biotin to catalyze DAB precipitation. The sections were counterstained with hematoxylin. For each xenografted skin, 2 sections were imaged with a light microscope at 20× magnification. Dermal IL-4+ cells were counted, and the dermal surface area was calculated in ImageJ imaging software, to yield a number of IL-4+ cells per mm².

IL-17A expression was analyzed by staining cryopreserved xenograft skin sections overnight at 4° C. with a rabbit anti-human IL-17A antibody (Ab79056, Abcam, 1:200) followed by species-specific fluorescent conjugated secondary antibody and DAPI counterstain to visualize the nuclei. For each xenografted skin, 2-3 sections were imaged with a fluorescence microscope at 20× magnification. Dermal IL-17A+ cells were counted, and the dermal surface area is calculated in ImageJ imaging software, to yield a number of IL-17A+ cells per mm².

IL-22 expression was analyzed by staining cryopreserved xenograft skin sections overnight at 4° C. with a rabbit anti-human IL-22 antibody (BS-2623R, ThermoFisher, 1:200) followed by species-specific fluorescent conjugated secondary antibody and DAPI counterstain to visualize the nuclei. For each xenografted skin, 2-3 sections were imaged with a fluorescence microscope at 20× magnification. Dermal IL-22+ cells were counted, and the dermal surface area is calculated in ImageJ imaging software, to yield a number of IL-22+ cells per mm².

IFN-γ expression was analyzed by staining Cryopreserved xenograft skin sections overnight at 4° C. with a rabbit anti-human IFN-γ antibody (Ab9657, Abcam, 1:100) followed by species-specific fluorescent conjugated secondary antibody and DAPI counterstain to visualize the nuclei. For each xenografted skin, 2-3 sections were imaged with a fluorescence microscope at 20× magnification. Dermal IFN-γ+ cells were counted, and the dermal surface area is calculated in ImageJ imaging software, to yield a number of IFN-γ+ cells per mm².

### Gene Expression Analysis in Humanized Model

RNA extraction was performed on OCT-embedded xenografts. OCT embedded xenografts from 55 mice were thawed over ice, then washed in PBS twice. Total RNA was extracted using a Precellys® Evolution Homogenisator and TRI Reagent® (Sigma-Aldrich). Briefly, each xenograft was homogenized for 6 cycles of 10 s at 6800 rpm and cooling on ice for 30 seconds and 1 mL of TRI Reagent® and Chloroform (250 μL) was added, sample was mixed, and centrifuged for 15 min at 12,500×g. Supernatant (~500 μL) containing total RNA was mixed with an equal volume of isopropanol for precipitation of RNA. Total RNA was pelleted by centrifugation at 12,500×g for 15 min, washed twice with 70% Ethanol and centrifugation at 8,000×g for 7 min, re-suspended in 20 μL RNase-free water. RNA was stored at −80° C. until analysis.

Quality control of the extracted RNA was measured using Tapestation Bioanalyzer and Qubit at the Nanostring Core facility at Heidelberg, Germany.

Nanostring nCounter© analysis system is based on digital detection of individual target molecules, directly labelled with molecular barcoding. Each probe consists of a color-coded Reporter and a Capture Probe, both with target-specific sequences (with a length of 50 nucleotides) covalently attached. The color-coded Reporter allows for the singularity of the target whereas the Capture Probe, conjugated to Biotin, allows the immobilization of the target onto a surface. With the application of voltage, and the creation of an electric field, the target molecules align, exposing the Reporter Probe in its full extension, and allowing the recognition of individual molecules. NanoString multiplexing was outsourced to the nCounter© Core Facility Heidelberg, Department of Human Molecular Genetics, Heidelberg University Hospital. Using the instrument nCounter© SPRINT.

In this study, nCounter© Human Immunology V2 Panel, which quantifies 594 genes, including housekeeping genes, was utilized. Purified RNA obtained from each specimen were hybridized overnight using Human Immunology V2 Profiling panel (NanoString Technologies, WA, USA) at 65° C. Further purification and binding of the hybridized probes to the optical cartridge were performed on nCounter© Prep Station, and the cartridge was scanned on nCounter Digital Analyzer. RCC files obtained from NanoString Digital Analyzer were imported into the nSolver™ 4.0.70 software (NanoString Technologies, WA, USA) and were checked for data quality using the default quality check settings. A “barcode” was used to determine the mRNA level, after which background correction was performed by subtracting the “mean+2 standard deviation” value of the negative controls from the raw counts and then the adjusted raw counts were normalized to the geometric mean of 12 determined housekeeping genes. Bioinformatics and statistical analyses were performed using nSolver™ Analysis Sofware, version 4.0.70 and the Human Immunology V2 Profiling Advanced Analysis module, including hierarchical clustering and volcano plots. As statistical significance with P adjusted value ≤0.05 could not be detected, we used raw p value ≤0.05 to analyze the data. Due to high cross reactivity with mouse genes (down to 85%) in the Human Immunology V2 Panel, we performed the analysis including all genes in the panel as well as excluding mouse genes with a cross reactivity of >85%. This is due to the information conveyed to us by the Nanostring Facility, that anything below 85% should not interfere with the analysis. To align the findings of this study with the current state of the art of AD gene profile, selected relevant genes in Atopic Dermatitis were selected).

Ex Vivo Human Skin Explant Stimulation with Th2-Polarizing Cocktail

Full-thickness human skin biopsies are achieved via punch-biopsy of residual surgical tissue. Biopsies are taken at a size of 3 mm² and transferred onto a mesh transwell in a 12 well plate. The dermis of the skin is submerged in specially fortified media, leaving the epidermis exposed to air. Biopsies are incubated in optimum conditions for a maximum of 24 hrs, then cultured in the presence of the Th2-polarizing inflammatory cocktail and the test articles for a further 24 hrs.

### Example 3: Analysis of the IL-15 Pathway in Samples from Atopic Dermatitis Patients

The expression of IL-15 and associated genes (“the IL-15 pathway”) is analyzed in various well characterized collections of samples or databases from Atopic Dermatitis patients and comparators; some of the samples are collected from patients who have received dupilumab and possible correlations will be made with the clinical outcome of the treatment. The objective of these analyses is to further characterize IL-15 pathway in AD, and possibly identify patient subsets that could preferentially benefit from a treatment with IL-15 inhibitors. Analyses using state of the art techniques, include:
Transcriptomics analysis from public databases
qPCR analysis from 30 samples of healthy and AD skin biopsies
Proteomics analysis (48 target genes) of 10 samples of healthy and AD skin biopsies
Immunohistochemistry analyses of 20 samples of healthy and AD skin biopsies
Single cell RNA sequencing of 10 samples of healthy and AD skin biopsies
Measure of IL-15 levels in 30 AD serum and healthy serum using an ultrasensitive technique.

For the analysis of data from public databases, the microarray dataset GSE130588, downloaded from the Gene Expression Omnibus (GEO) public database which contains data from human Atopic Dermatitis skin and healthy skin samples as described in Guttman-Yassky et al. 2019, J. Allergy Clin. Immunol., 143, 155-172 was used.

Biopsy specimens were collected from lesional and non-lesional skin in a randomized, placebo-controlled, double-blind, phase 2 trial (ClinicalTrials.gov: NCT01979016) conducted at 5 medical centers in the United States and Canada to assess the efficacy and safety of dupilumab compared with placebo in patients with moderate-to-severe Atopic Dermatitis. Patients received weekly subcutaneous injections of 200 mg of dupilumab or placebo after a 400 mg loading dose or placebo on day 1, for a total of 16 weeks. RNA was extracted, followed by hybridization to Affymetrix Human U133Plus 2.0 gene arrays (Affymetrix, Santa Clara, Calif). The original CEL-format file was converted into an expression profile format with the open-source package Affy, available from the R project website (https://cran.r-project.org). The data were normalized with the median method. Normalized gene expression values were calculated to compare IL-15 expression between patient groups.

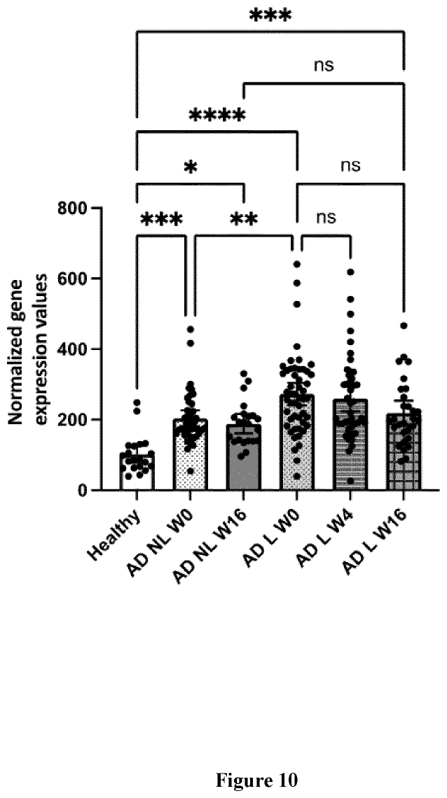

It is observed that IL-15 was significantly more expressed in both non-lesional (NL) and lesional (L) skin of Atopic Dermatitis (FIG. 10), in agreement with what was reported before (Karlen and Simon, 2020, Int. Arch. Allergy Immunol., 181, 417-421). IL-15 expression was also significantly higher in lesional versus non-lesional Atopic Dermatitis skin. It was also observed that, while Dupilumab treatment slightly reduced IL-15 expression in lesional Atopic Dermatitis Skin, its effect was not significant. In fact, at week 16 (W16) of Dupilumab treatment, IL-15 expression remains significantly higher in both lesional and non-lesional Atopic Dermatitis skin, compared to healthy skin. These results suggest that the IL-15 pathway is not fully inhibited by treatment with anti-IL-4R antibody Dupilumab in patients, as already observed in the mouse humanized model (FIG. 9) and therefore further supports that an anti-IL-15 therapy might be advantageously administered to patients not responding to anti-IL-4R antibody, or in combination with those or other treatments.

### Example 4: Generalization of the Effects of Anti-IL15 Antibody Hu-BE29-2 to the Other Antibodies Sharing the Same Binding Epitope

The effects described above can be reasonably generalized described to other antibodies sharing the same binding epitope such as those described in WO 2016/001275, in particular hu-BE29-10 & 24.

The same epitope specificity of those anti human IL 15 antibodies as for hu-BE29-2 has been confirmed as described below.

A library of structured peptides of IL-15 into single loops, double loops, triple loops, sheet-like folds, helix-like folds, and combinations thereof was synthesized using Pepscan's proprietary Chemically Linked Peptides on Scaffolds (CLIPS) technology (Timmerman 2007, Journal of Molecular recognition, https://doi.org/10.1002/jmr.846). CLIPS templates were coupled to side-chain thiol groups of cysteine residues and peptide arrays were prepared. The binding of antibodies hu-BE29-2, hu-BE29-10 and hu-BE29-24 to each of the synthesized peptides was tested by an ELISA technique where color development was quantified with a charge coupled device (CCD) camera and an image processing system. Results showed that antibodies hu-BE29-2, hu-BE29-10 and hu-BE29-24 all bind to the epitope in IL-15 of a sequence consisting in the amino acids 83 to 93 of SEQ ID NO:1.

LIST OF SEQUENCES
Human mature IL-15 (Homo sapiens), GenBank: CAA71044.1
SEQ ID NO: 1
MDFQVQIFSFLLISASVIMSRANWVNVISDLKKIEDLIQSMHIDATLYTESDVHPSCK
VTAMKCFLLELQVISLESGDASIHDTVENLIILANNSLSSNGNVTESGCKECEELEEK
NIKEFLQSFVHIVQMFINTS
Cytokine modulator peptide from WO 2015/089217, BNZ132 (AKA
BNZ-1, BNZ-13, YT-033, EQ-101)
SEQ ID NO: 2
IKEFLQRFIHIVQSIINTS
Gamma-cytokine modulator peptide from US 2019/0070263
SEQ ID NO: 3
PKEFLERFVHLVQMFIHQSLS
IL-15 inhibitor peptide US7, 736,638B2 SEQ ID NO: 4
SEQ ID NO: 4
KVTAMKCFLL
huVH1 from WO 2016/001275
SEQ ID NO: 5
EVRLMASGGGLVQPGGSLRLSCAASEFTESNYAMSWVRQAPGKGLEWVATISRGGDYT
YYPDSVKGRFTISRDNSKNTLYLQMNSLRAEDTAVYYCARRVSMITGGWAMDYWGQGT
LVTVSS
huVH2 from WO 2016/001275
SEQ ID NO: 6
EVQLVESGGGLVQPGGSLRLSCAASEFTFSNYAMSWVRQAPGKGLEWVATISRGGDYT
YYPDSVKGRFTISRDNSKNTLYLQMNSLRAEDTAVYYCARRVSMITGGWAMDYWGQGT
LVTVSS
huVH8 from WO 2016/001275
SEQ ID NO: 7
EVRLMASGGGLVQPGGSLRLSCAASEFTFSNYAMSWVRQAPGKGLEWVATISRGGDYT
YYPDTVKGRFTISRDNSKNTLYLQMNSLRAEDTAVYYCARRVSMITGGYAMDYWGQGT
LVTVSS
huVH18 from WO 2016/001275
SEQ ID NO: 8
EVQLVESGGGLVQPGGSLRLSCAASEFTESNYAMSWVRQAPGKGLEWVATISRGGDYT
YYPDSVKGRFTISRDNSKNTLYLQMNSLRAEDTAVYYCARRVSMITGGYAMDYWGQGT
LVTVSS
huVL1 from WO 2016/001275
SEQ ID NO: 9
DVVMTQSPLSLPVTLGQPASISCRSSQSIVDITGNTYLEWYQQRPGQSPRLLIYKVEN
RFSGVPDRFSGSGSGTDFTLKISRVEAEDVGVYYCFQDSFVPYTFGQGTKLEIK
Heavy Chain from 146B7 aka AMG 714 (WO 2005/044303)
SEQ ID NO: 10
MGWTLVFLFLLSVTAGVHSEVQLVQSGAEVKKPGESLKISCKVSGYFFTTYWIGWVRQ
MPGKGLEYMGIIYPGDSDTRYSPSFQGQVTISADKSI STAYLQWSSLKASDTAMYYCA
RGGNWNCFDYWGQGTLVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKVD
KRVEPKSCDKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYKCKVSNKAL
PAPIEKTISKAKGQPREPQVYTLPPSREEMTKNQVSLTCLVKGFYPSDIAVEWESNGQ
PENNYKTTPPVLDSDGSFFLYSKLTVDKSRWQQGNVFSCSVMHEALHNHYTQKSLSLS
PGK
Light Chain from 146B7 aka AMG 714 (WO 2005/044303)
SEQ ID NO: 11
MVSSAQFLGLLLLCFQGTRCEIVLTQSPGTLSLSPGERATLSCRASQSVSSSYLAWYQ
QKPGQAPRLLIYGASRRATGIPDRESGSGSGTDFTLTISRLEPEDFAVYYCQRYGSSH
TFGQGTKLEISRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLS
SPVTKSENRGEC
Variable heavy chain region from 70a WO 2018/119246
SEQ ID NO: 12
QVQLQESGPGLVKPSGTLSLTCAVSGGSISSSNWWSWVRQPPGKGLEWIGEIYHYGYT
NYNPSLKSRVTISVDKSKNQFSLKLSSVTAADTAVYYCAREGIGWPSFDYWGQGTLVT
VSS
Variable light chain region from 70a WO 2018/119246
SEQ ID NO: 13
SSELTQDPAASVALGQTVRITCQGDTLRSYYASWYQQKPGQAPILVIYGKNNRPSGIP
DRFSGSSSGNTASLTITGAQAEDEADYYCNSRDLSGKNLVFGGGTKLTVL
Variable heavy chain region from huABC2 WO 2011/127324
SEQ ID NO: 14
MKLWLNWVFLLTLLHGIQCEVQLVESGGGLVQPGGSLRLSCAASGFTFSDFYMEWVRQ
APGKGLEWIAAASRNKANDYTTEYSASVKGRFIVSRDDSKNSLYLQMNSLKTEDTAVY
YCARSYYRYDGMDYWGQGTTVTVSS
Variable light chain region from huABC2 WO 2011/127324
SEQ ID NO: 15
MDFQVQIFSFLLISASVIVSRGEIVLTQSPATLSLSPGERATLSCSAISSVSYMYWYQ
QKPGQAPRLLIYDTSNLVSGVPARFSGSGSGTDYTLTISSLEPEDFAYYYCQQWNTYP
YTFGGGTKVEIK
Heavy Chain from huBE29-2 (WO 2016/001275)
SEQ ID NO: 16
EVQLVESGGGLVQPGGSLRLSCAASEFTFSNYAMSWVRQAPGKGLEWVATISRGGDYT
YYPDSVKGRFTISRDNSKNTLYLQMNSLRAEDTAVYYCARRVSMITGGWAMDYWGQGT
LVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDKTHTCP
PCPAPELLGGPSVFLFPPKPKDTLMISRTPEVTCVVVDVSHEDPEVKENWYVDGVEVH
NAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
Light Chain from huBE29-2 (WO 2016/001275)
SEQ ID NO: 17
DVVMTQSPLSLPVTLGQPASISCRSSQSIVDITGNTYLEWYQQRPGQSPRLLIYKVEN
RFSGVPDRESGSGSGTDFTLKISRVEAEDVGVYYCFQDSFVPYTFGQGTKLEIKRTVA
APSVFIFPPSDEQLKSGTASVVCLLNNFYPREAKVQWKVDNALQSGNSQESVTEQDSK
DSTYSLSSTLTLSKADYEKHKVYACEVTHQGLSSPVTKSENRGEC
Heavy Chain from huBE29-10 (WO 2016/001275)
SEQ ID NO: 18
EVRLMASGGGLVQPGGSLRLSCAASEFTFSNYAMSWVRQAPGKGLEWVATISRGGDYT
YYPDTVKGRFTISRDNSKNTLYLQMNSLRAEDTAVYYCARRVSMITGGYAMDYWGQGT
LVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDKTHTCP
PCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK
Heavy Chain from huBE29-24 (WO 2016/001275)
SEQ ID NO: 19
EVQLVESGGGLVQPGGSLRLSCAASEFTFSNYAMSWVRQAPGKGLEWVATISRGGDYT
YYPDSVKGRFTISRDNSKNTLYLQMNSLRAEDTAVYYCARRVSMITGGYAMDYWGQGT
LVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKRVEPKSCDKTHTCP
PCPAPELLGGPSVFLFPPKPKDTLMISRTPEVTCVVVDVSHEDPEVKENWYVDGVEVH
NAKTKPREEQYNSTYRVVSVLTVLHQDWLNGKEYKCKVSNKALPAPIEKTISKAKGQP
REPQVYTLPPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

1. An interleukin-15 inhibitor for use in the treatment of atopic dermatitis.

2. An interleukin-15 inhibitor for use according to claim 1, wherein said interleukin-15 inhibitor is a small molecule.

3. An interleukin-15 inhibitor for use according to claim 2, wherein said interleukin-15 inhibitor is of Formula (I) 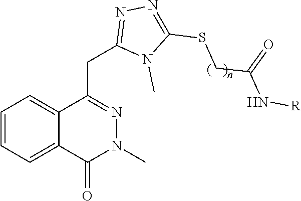
wherein R is an optionally substituted phenyl and n is an integer selected from 1 to 10.

4. An interleukin-15 inhibitor for use according to claim 1, wherein said interleukin-15 inhibitor is a peptide.

5. An interleukin-15 inhibitor for use according to claim 4, wherein said interleukin-15 inhibitor is a peptide comprising a sequence selected from SEQ ID NO: 2, SEQ ID NO: 3, and SEQ ID NO: 4 or a fragment or variant thereof.

6. An interleukin-15 inhibitor for use according to claim 1, wherein said interleukin-15 inhibitor is an anti-IL-15 antibody.

7. An interleukin-15 inhibitor for use according to claim 1 or 6, wherein said interleukin-15 inhibitor is an isolated antibody binding IL-15 or an antigen-binding fragment thereof comprising:
(1) a heavy chain variable region of SEQ ID NO: 5 or any variant thereof wherein said variant has the amino acid sequence of SEQ ID NO: 5 except that 1, 2, 3 or 4 amino acids are substituted by a different amino acid, wherein substitutions are selected among:
(iv) arginine (R) at position H3 (VH RH3) substituted by glutamine (Q), methionine (M) at position H5 (VH MH5) substituted by valine (V), alanine (A) at position H6 (VH AH6) substituted by glutamic acid (E), alanine (A) at position H49 (VH AH49) substituted by serine (S), within the heavy chain variable framework region,
(v) aspartic acid (D) at position H61 (VH DH61) substituted by glutamic acid (E), serine (S) at position H62 (VH SH62) substituted by threonine (T), within the heavy chain CDR2, and
(vi) methionine (M) at position H98 (VH MH98) substituted by leucine (L), phenylalanine (F), isoleucine (I), or alanine (A), tryptophan (W) at position H100C (VH WH100C) substituted by tyrosine (Y), phenylalanine (F) or alanine (A), methionine (M) at position H100E (VH MH100E) substituted by leucine (L), phenylalanine (F) or isoleucine (I), within the heavy chain CDR3;
and
(2) a light chain variable region of SEQ ID NO: 9.

8. An interleukin-15 inhibitor for use according to claim 1 or 6, wherein said interleukin-15 inhibitor is an isolated antibody binding IL-15 or an antigen-binding fragment thereof comprising:
(1) a heavy chain variable region of SEQ ID NO: 6 or any variant thereof wherein said variant has the amino acid sequence of SEQ ID NO: 6 except that 1, 2, 3 or 4 amino acids are substituted by a different amino acid and
(2) a light chain variable region of SEQ ID NO: 9.

9. An interleukin-15 inhibitor for use according to claim 7, wherein said interleukin-15 inhibitor is an isolated antibody binding IL-15 or an antigen-binding fragment thereof, comprising:
(3) a heavy chain variable region of amino acid sequence of SEQ ID NO: 5 having substitutions in said sequence of SEQ ID NO: 5 selected among:
(ii) VH RH3 is substituted by glutamine (Q), VH MH5 is substituted by valine (V), VH AH6 is substituted by glutamic acid (E),
(iv) VH SH62 is substituted by threonine (T), and
(v) VH WH100C is substituted by tyrosine (Y); and
(4) a light chain variable region of amino acid sequence of SEQ ID NO: 9.

10. An interleukin-15 inhibitor for use according to claim 1 or 6, wherein said interleukin-15 inhibitor is an isolated antibody binding IL-15 or an antigen-binding fragment thereof, comprising:
(1) a heavy chain variable region selected from: SEQ ID NO: 5, SEQ ID NO: 6, SEQ ID NO: 7 and SEQ ID NO: 8, and
(2) a light chain variable region of SEQ ID NO: 9.

11. An interleukin-15 inhibitor for use according to claim 1 or 6, wherein said interleukin-15 inhibitor selected from an anti-IL-15 antibody comprising:
a heavy chain of SEQ ID NO: 10 and a light chain of SEQ ID NO: 11 or a variant or fragment thereof; or
a variable heavy chain region of SEQ ID NO: 12 or a variant or fragment thereof and a variable light chain region of SEQ ID NO: 13 or a variant or fragment thereof; or
a variable heavy chain region of SEQ ID NO: 14 or a variant or fragment thereof and a variable light chain region of SEQ ID NO: 15 or a variant or fragment thereof; or
a heavy chain of SEQ ID NO: 16 and a light chain of SEQ ID NO: 17 or a variant a variant or fragment thereof; or
a heavy chain of SEQ ID NO: 18 and a light chain of SEQ ID NO: 17 or a variant a variant or fragment thereof, or
a heavy chain of SEQ ID NO: 19 and a light chain of SEQ ID NO: 17 or a variant a variant or fragment thereof.

12. A pharmaceutical composition comprising an interleukin-15 inhibitor and an agent useful in the prevention and/or treatment of atopic dermatitis and a pharmaceutically acceptable carrier, diluent or excipient thereof.

13. A pharmaceutical composition according to claim 12 further comprising a co-agent useful in the prevention and/or treatment of atopic dermatitis such as at least one anti-IL-4R antibody or at least one corticosteroid or a mixture thereof.

14. A pharmaceutical composition according to claim 12 or 13, further comprising at least one anti-IL-4R antibody, such as dupilumab, an anti-IL-13 such as tralokinumab, a JAK inhibitor such as abrocitinib, upadacitinib or ruxolitinib.

15. A pharmaceutical composition according to claim 12 or 13, further comprising at least one corticosteroid, such as dexamethasone.

16. A method of preventing and/or treating atopic dermatitis in a subject, said method comprising administering in a subject in need thereof a therapeutically effective amount of an interleukin-15 inhibitor or pharmaceutical composition thereof.

17. A method according to claim 15, wherein said interleukin-15 inhibitor is at least one as described in any one of claims 1 to 11.

18. A method according to claim 15 or 16, wherein said interleukin-15 inhibitor is administered in the form of a formulation according to any one of claims 12 to 15.
